(19) United States
(12) Patent Application Publication
(10) Pub. No.: US 2014/0186363 A1
(43) Pub. Date: Jul. 3, 2014

(54) MAGEA3 BINDING ANTIBODIES

(71) Applicants: Christoph Esslinger, Zurich (CH); Michael Höcker, Zurich (CH); Martin Treder, Zurich (CH); Alexander Knuth, Zurich (CH); Elke Jaeger, Mainz-Kostheim (DE)

(72) Inventors: Christoph Esslinger, Zurich (CH); Michael Höcker, Zurich (CH); Martin Treder, Zurich (CH); Alexander Knuth, Zurich (CH); Elke Jaeger, Mainz-Kostheim (DE)

(73) Assignees: UNIVERSITY OF ZURICH, Zurich (CH); CT ATLANTIC LTD., Schlieren (CH)

(21) Appl. No.: 14/123,584

(22) PCT Filed: May 23, 2012
(86) PCT No.: PCT/EP2012/059642

§ 371 Date: Dec. 18, 2013

(30) Foreign Application Priority Data
Jun. 3, 2011 (EP) 11168699.4

(51) Int. Cl. C07K 16/18

(52) U.S. Cl. CPC C07K 16/18
USPC 424/142.1; 530/388.15

(57) ABSTRACT

The present invention relates to MAGEA3 binding antibodies.

### BACKGROUND OF THE INVENTION

1. Field of the Invention

The present invention relates to MAGEA3 binding antibodies or binding fragments thereof. The present invention further relates to the use of such MAGEA3 binding antibodies or binding fragments thereof, in particular hyper-proliferative diseases and methods for treating diseases, in particular hyper-proliferative diseases with such combinations.

2. Related Background

In cancer therapy, it is a general aim to treat the afflicted tissues as efficiently and selectively as possible. Therapeutic monoclonal antibodies have been conceived as a class of pharmaceutically active agents, which should allow tumor selective treatment, by targeting tumor selective antigens or epitopes.

However, in some cancers, for example those associated with human growth factor receptors such as HER-2 R or EGFR, epitopes targeted by therapeutic antibodies are also found on normal tissues explaining adverse side effects upon antibody administration or peripheral sink effects in the pharmacokinetic behavior of such antibodies.

The analogous situation holds true by applying systemically active immune-stimulatory drugs or antibodies applied to stimulate a natural immune response to fight cancer. Such immune-stimulants are for example activators of the innate immune system such as activators of TLR-7 or TLR-9 receptors.

Monoclonal antibodies have nevertheless enjoyed increasing acceptance as therapeutic tools for treating cancer over the past decades. The advent of chimeric antibodies and humanized antibodies significantly contributed to the success of monoclonal therapeutic antibodies as these second and third generation monoclonal antibodies showed improved side-effect profiles compared to the original mice-derived monoclonal antibodies in view of their reduced immunogenicity.

Despite the proven therapeutic efficacy of humanized antibodies, there is an interest in fully human antibodies. However, production thereof is still prone to technical difficulties. For example, generating fully human antibodies in mice in which the antibody encoding genomic regions have been replaced by the human counterpart remains burdensome. Alternative approaches such as phage display lack the natural variability and complexity of the human immune system.

There is thus continuing need for therapeutic monoclonal antibodies which allow for a (tumor) localized mode of action and which have an increased chance of meeting regulatory approval. Moreover, there is a wish for cancer therapies in general which allow for improved efficacy.

### SUMMARY OF THE INVENTION

It is an objective of the present invention to provide pharmaceutically active agents, which can be used as therapeutic and/or diagnostic tool for treating/diagnosing human diseases including hyper-proliferative diseases such as cancer. In particular, it is an objective of the present invention to provide combinations of pharmaceutically active agents, which can be used to selectively treat hyper-proliferative diseases by ensuring a localized immune reaction in the afflicted tissue.

Further, it is an objective of the present invention to provide methods of treating and/or diagnosing patients suffering or suspected of suffering from e.g. from hyper-proliferative diseases such as cancer by making use of such pharmaceutically active agents and such combinations of pharmaceutically active agents.

These and other objectives as they will become apparent from the ensuing description hereinafter are solved by the subject matter of the independent claims. Some of the preferred embodiments of the present invention form the subject matter of the dependent claims. Yet other embodiments of the present invention may be taken from the ensuing description.

In a first aspect, the invention relates generally to isolated monoclonal MAGEA3 binding antibodies or binding fragments thereof.

In one embodiment of the first aspect, the invention thus relates to pharmaceutical compositions comprising such isolated monoclonal MAGEA3 binding antibodies or binding fragments thereof.

In another embodiment of the first aspect, the invention thus relates to diagnostic compositions comprising such isolated monoclonal MAGEA3 binding antibodies or binding fragments thereof.

Such MAGEA3 binding antibodies or binding fragments thereof may be monoclonal chimeric, humanized or human antibodies or binding fragments thereof. Patient-derived, human, monoclonal antibodies may be preferred.

In another embodiment such MAGEA3 binding antibodies or binding fragments thereof preferentially bind to MAGEA3 over other MAGE isoforms such as MAGE-MAGEA1, MAGEA4, MAGEA10 and/or optionally even MAGEA2.

Preferred exemplary MAGEA3 binding antibodies or fragments thereof may comprise a variable heavy chain and/or a variable light chain of the exemplary antibodies 122G3, 32H2, 34G9 or 102G10 or a variable heavy chain and/or a variable light chain having at least 80% sequence identity with the variable heavy chain and/or variable light chain of the exemplary antibodies 122G3, 32H2, 34G9 or 102G10.

Other preferred exemplary MAGEA3 binding antibodies or fragments thereof may comprise at least one, two or all three of complementary determining regions (CDRs) of the exemplary antibodies 122G3, 32H2, 34G9 or 102G10 within their variable heavy chain and/or variable light chain. Such antibodies may also comprise CDRs within their variable heavy chain and/or variable light chain having at least 80% sequence identity with the CDRs of the exemplary antibodies 122G3, 32H2, 34G9 or 102G10.

The present invention further relates to pharmaceutical compositions comprising such specific MAGEA3 binding antibodies or binding fragments thereof for use in treating hyper-proliferative disease, in particular tumors, which express MAGEA3.

The present invention further relates to the use of such specific MAGEA3 binding antibodies or binding fragments thereof in the manufacture of a medicament for treating hyper-proliferative disease, in particular tumors, which express MAGEA3.

The present invention further relates to methods of treating hyper-proliferative disease, in particular tumors, which express MAGEA3 by administering to patients such specific MAGEA3 binding antibodies or binding fragments thereof. Such hyperproliferative diseases preferably include melanoma, breast cancer, ovarian cancer, non-small cell lung cancer, multiple myeloma and/or pancreatic cancer.

In one embodiment of this first aspect of the present invention, the pharmaceutical compositions comprising the MAGEA3 binding antibodies or binding fragments thereof do not comprise a compound capable of activating the immune system.

In another embodiment of this first aspect of the present invention, the pharmaceutical compositions comprise the MAGEA3 binding antibodies or binding fragments as the sole pharmaceutically active agent.

In other embodiments of the first aspect of the invention, the MAGEA3 binding antibodies or binding fragments thereof as described herein are used as a diagnostic tool, e.g. for diagnosing patients suffering from hyperproliferative diseases as mentioned herein. It can be preferred to use such antibodies to diagnose the occurrence and/or development of e.g. hyperproliferative diseases, which express MAGEA3 and/or MAGEA6. Such hyperproliferative diseases may include melanoma, breast cancer, ovarian cancer, non-small cell lung cancer, multiple myeloma and/or pancreatic cancer.

In one embodiment, the present invention thus relates to a diagnostic composition comprising the MAGEA3 binding antibodies or binding fragments described herein. Such diagnostic compositions may be for use in diagnosing occurrence and/or development of e.g. hyperproliferative diseases, which express MAGEA3 and/or MAGEA6. Such hyperproliferative diseases may include melanoma, breast cancer, ovarian cancer, non-small cell lung cancer, multiple myeloma and/or pancreatic cancer.

In another embodiment, the invention relates to MAGEA3 binding antibodies or binding fragments thereof as described herein for use in diagnosing hyperproliferative diseases. These diseases may express MAGEA3 and/or MAGEA6. Such hyperproliferative diseases may include melanoma, breast cancer, ovarian cancer, non-small cell lung cancer, multiple myeloma and/or pancreatic cancer.

In another embodiment, the present invention relates to the use of MAGEA3 binding antibodies or binding fragments thereof as described herein in the manufacture of a composition and/or medicament for diagnosing hyperproliferative diseases. These diseases may express MAGEA3 and/or MAGEA6. Such hyperproliferative diseases may include melanoma, breast cancer, ovarian cancer, non-small cell lung cancer, multiple myeloma and/or pancreatic cancer.

In yet another embodiment, the present invention relates to a method of diagnosing a hyperproliferative disease in a human or animal being by using MAGEA3 binding antibodies or binding fragments thereof as described herein. These diseases may express MAGEA3 and/or MAGEA6. Such hyperproliferative diseases may include melanoma, breast cancer, ovarian cancer, non-small cell lung cancer, multiple myeloma and/or pancreatic cancer.

In a second aspect, the invention relates to a combination of at least one tumor associated antigen (TAA) binding antibody or binding fragment thereof and at least one compound capable of activating the immune system.

In one embodiment of the second aspect, the present invention relates to a pharmaceutical composition comprising at least one tumor-associated antigen (TAA) binding antibody or binding fragment thereof and at least one compound capable of activating the immune system.

As will become apparent from the ensuing description, such TAA binding antibodies or binding fragments thereof preferably bind to CT antigens with NY-ESO-1 or MAGEA3 being examples thereof. Such antibodies or binding fragments thereof may be monoclonal chimeric, humanized or human antibodies or binding fragments thereof. Patient-derived, human, monoclonal antibodies may be preferred.

Preferred exemplary MAGEA3 binding antibodies or fragments thereof may be the MAGEA3 binding antibodies or binding fragments thereof as mentioned hereinafter. Exemplary NY-ESO-1 binding antibodies or binding fragments thereof may be those mentioned in EP 11 150 527.7.

As will become apparent from the ensuing description, compounds capable of activating the immune response may preferably be selected from at least one natural stimulant or at least co-stimulant of the immune system, agonistic activator of natural stimulants or at least co-stimulants of the immune system or at least one antagonistic effector of natural inhibitors or at least co-inhibitors of the immune system as described hereinafter. Some preferred exemplary representatives are CD40L, anti-CD40 agonistic antibodies such as CP-870,893 and SGN-40 and anti-CTLA4 antagonistic antibodies such as Tremelimumab and Ipilimumab.

Preferred exemplary embodiments thus relate to pharmaceutical compositions comprising (i) the MAGEA3 binding antibodies or fragments thereof as mentioned hereinafter, and (ii) CD40L, or anti-CD40 agonistic antibodies such as CP-870,893 and SGN-40, or anti-CTLA4 antagonistic antibodies such as Tremelimumab and Ipilimumab.

In a preferred embodiment, the pharmaceutical composition may comprise (i) a TAA binding antibody or binding fragment such as the MAGEA3 binding antibodies or binding fragments thereof as mentioned hereinafter, (ii) at least one natural stimulant or at least co-stimulant of the immune system, or at least one agonistic activator of natural stimulants or at least co-stimulants of the immune system and (iii) at least one antagonistic effector of natural inhibitors or at least co-inhibitors of the immune system as described hereinafter. Some preferred exemplary embodiments relate to pharmaceutical compositions comprising (i) the aforementioned MAGEA3 binding antibodies or fragments thereof, (ii) CD40L or anti-CD40 agonistic antibodies such as CP-870,893 or SGN-40 and (iii) anti-CTLA4 antagonistic antibodies such as Tremelimumab and Ipilimumab.

In another embodiment of the second aspect of the invention, the aforementioned pharmaceutically active agents, i.e. the TAA binding antibodies or fragments thereof such as the MAGEA3 binding antibodies or binding fragments thereof as mentioned hereinafter and the compounds capable of stimulating the immune system are not combined within a single pharmaceutical composition but actually are presented in form of a kit consisting of various pharmaceutical compositions wherein the active agents are split at least to some extent between the various pharmaceutical compositions.

For example, one pharmaceutical composition of such a kit may comprise a TAA binding antibody or binding fragment thereof such as the MAGEA3 binding antibodies or binding fragments thereof as mentioned hereinafter while a second pharmaceutical composition may comprise at least one agonistic activator of natural stimulants or at least co-stimulants of the immune system such as anti-CD40 agonistic antibodies or at least one antagonistic effector of natural inhibitors or at least co-inhibitors of the immune system such as anti-CTLA4 antagonistic antibodies.

In embodiments where the kit comprises a TAA binding antibody or binding fragment thereof such as the MAGEA3 binding antibodies or binding fragments thereof as mentioned hereinafter and both at least one agonistic activator of natural stimulants or at least co-stimulants of the immune system such as anti-CD40 agonistic antibodies, and at least one antagonistic effector of natural inhibitors or at least co-inhibitors of the immune system such as anti-CTLA4 antagonistic antibodies, one pharmaceutical composition of such a kit may comprise a TAA binding antibody or binding fragment thereof such as the MAGEA3 binding antibodies or binding fragments thereof as mentioned hereinafter, while a second pharmaceutical composition may comprise at least one agonistic activator of natural stimulants or at least co-stimulants of the immune system such as anti-CD40 agonistic antibodies and a third pharmaceutical composition may comprise at least one antagonistic effector of natural inhibitors or at least co-inhibitors of the immune system such as anti-CTLA4 antagonistic antibodies. In alternative thereof, the second pharmaceutical composition may comprise both at least one agonistic activator of natural stimulants or at least co-stimulants of the immune system such as anti-CD40 agonistic antibodies and at least one antagonistic effector of natural inhibitors or at least co-inhibitors of the immune system such as anti-CTLA4 antagonistic antibodies.

Such kits allow treatment of patients by subsequent and/or at least partially simultaneous administration of the various pharmaceutical preparations, which form the kit and may thus enable a timely optimized treatment regimen of the above-mentioned combinations.

The present invention also relates to a combination of at least one tumor associated antigen (TAA) binding antibody or binding fragment thereof such as the MAGEA3 binding antibodies or binding fragments thereof as mentioned hereinafter and at least one compound capable of activating the immune system for use in treating a disease such as a hyper-proliferative disease. The TAA binding antibody or binding fragments thereof such as the MAGEA3 binding antibodies or binding fragments thereof as mentioned hereinafter and the at least one compound capable of activating the immune system may be selected as described hereinafter.

As is described hereinafter, the combinations of active agents in accordance with the invention, i.e. TAA binding antibodies or fragments thereof such as the MAGEA3 binding antibodies or binding fragments thereof as mentioned hereinafter and compounds which are capable of stimulating the immune system, may provide improved efficacy if patients are subjected to cytotoxic treatment prior to, simultaneous with or subsequent to administration of the aforementioned pharmaceutical compositions, kits or combinations comprising such active agents. It may be preferred that patients receive such cytotoxic treatment prior to or simultaneous with administration of the aforementioned pharmaceutical compositions, kits or combinations comprising such active agents.

If such cytotoxic treatment comprises administration of cytotoxic agents, such cytotoxic agents may be included in the pharmaceutical compositions or kits in accordance with the invention. One exemplary preferred representative of such cytotoxic agents is 5-fluoro uracil (5-FU).

In another embodiment of the second aspect of the invention, the pharmaceutical compositions and kits in accordance with the invention may be used to treat patients suffering or being suspected to be prone to hyper-proliferative diseases, such as cancer.

Preferably, the pharmaceutical compositions and kits in accordance with the invention may be used to treat patients suffering or being suspected to be prone to cancers, which are characterized by the expression of TAAs such as cancers being characterized by the expression of CT-antigens such as MAGEA3.

If the TAA binding antibody or binding fragment thereof which is comprised within the pharmaceutical compositions and kits in accordance with the invention is a MAGEA3 binding antibodies or binding fragments thereof as mentioned hereinafter, the treatment may be of cancers such as melanoma, breast cancer, ovarian cancer, non-small cell lung cancer, multiple myeloma and/or pancreatic cancer.

Another embodiment of the second aspect of the present invention thus also relates to a medicament for use in treating a patient wherein a pharmaceutical composition or a kit as described hereinafter is used. TAA binding antibodies or binding fragments may preferably be MAGEA3 binding antibodies or binding fragments thereof and compounds capable of activating the immune response may preferably be selected from at least one natural stimulant or at least co-stimulant of the immune system, agonistic activator of natural stimulants or at least co-stimulants of the immune system or at least one antagonistic effector of natural inhibitors or at least co-inhibitors of the immune system as described hereinafter. In some embodiments, a combination of the MAGEA3 binding antibodies or binding fragments thereof as mentioned herein, anti-CD40 agonistic antibodies such as CP-870,893 and SGN-40 and/or anti-CTLA4 antagonistic antibodies such as Tremelimumab and Ipilimumab are envisaged.

Such medicaments may be used for patients who are subjected to cytotoxic treatment prior to, simultaneous with or subsequent to administration of such medicaments. In one embodiment the cytotoxic treatment may include chemotherapy.

Such medicaments may in particular be used for treatment of hyper-proliferative disease such as cancer.

The present invention in an embodiment of the second aspect of the invention also relates to the use of a pharmaceutical composition or a kit as described hereinafter in the manufacture of a medicament for treating a patient. TAA binding antibodies or binding fragments may preferably be MAGEA3 binding antibodies or binding fragments thereof and compounds capable of activating the immune response may preferably be selected from at least one natural stimulant or at least co-stimulant of the immune system, agonistic activator of natural stimulants or at least co-stimulants of the immune system or at least one antagonistic effector of natural inhibitors or at least co-inhibitors of the immune system as described hereinafter. In some embodiments, a combination of the MAGEA3 binding antibodies or binding fragments thereof as mentioned herein, anti-CD40 agonistic antibodies such as CP-870,893 and SGN-40 and/or anti-CTLA4 antagonistic antibodies such as Tremelimumab and Ipilimumab are envisaged.

Such medicaments may be used for patients, which are subjected to cytotoxic treatment prior to, simultaneous with or subsequent to administration of such medicaments. In one embodiment the cytotoxic treatment may include chemotherapy.

Such medicaments may in particular be used for treatment of hyper-proliferative disease such as cancer.

The present invention also relates to a method of treating a patient by administering a pharmaceutical composition or a kit as described hereinafter to the patient. TAA binding antibodies or binding fragments may preferably be MAGEA3 binding antibodies or binding fragments thereof and compounds capable of activating the immune response may preferably be selected from at least one natural stimulant or at least co-stimulant of the immune system, agonistic activator of natural stimulants or at least co-stimulants of the immune system or at least one antagonistic effector of natural inhibitors or at least co-inhibitors of the immune system as described hereinafter. In some embodiments, a combination of the MAGEA3 binding antibodies or binding fragments thereof as mentioned herein, anti-CD40 agonistic antibodies such as CP-870,893 and SGN-40 and/or anti-CTLA4 antagonistic antibodies such as Tremelimumab and Ipilimumab are envisaged.

Such methods may be considered for patients, who are subjected to cytotoxic treatment prior to, simultaneous with or subsequent to administration of such medicaments. In one embodiment the cytotoxic treatment may include chemotherapy.

Such methods may be considered for treatment of hyper-proliferative disease such as cancer.

### FIGURE LEGENDS

FIG. 1 Binding of recombinant human monoclonal antibodies to epitopes on MAGEA3. Antibodies 122G3, 32H2, 34G9 and 102G10 were analyzed by ELISA using plates coated with overlapping peptides spanning the entire MAGEA3-protein. Antibodies 122G3, 32H2, 34G9 or 102G10 bound to one or several of the MAGEA3 overlapping peptides (A-D).

FIG. 2 Immunoprecipitation of MAGEA3 by recombinant human monoclonal antibodies. Antibodies 122G3 and 32H2 were incubated with lysates of SK-MEL-37 cells endogenously expressing MAGEA3 (lane B). Antibody precipitated MAGEA3 protein was detected by Western blot using a MAGEA3 specific antibody. As controls, antibody alone (lane A) or antibody incubated with lysate of HEK 293T cells not expressing MAGEA3 (lane C) was used.

FIG. 3 Recognition of MAGEA, but not of MAGEA4 by recombinant human monoclonal antibodies. Human antibodies 122G3, 32H2, 34 G9 and 102G10 were used to detect recombinant MAGEA3-(lane A) and recombinant MAGEA4-protein (lane B) in Western Blot analysis.

FIG. 4 EC50 determination of recombinant human monoclonal antibodies. MAGEA3-ELISA using plates coated with recombinant MAGEA3 and serial dilutions of the human monoclonal antibodies 122G3, 32H2, 34G9 and 102G10 (A-D) was carried out. EC50 values are indicated in the figure.

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody, which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense. If a specific meaning is conveyed to certain terms, definitions of terms will be given in the following in the context of which the terms are used.

The term “Tumor Associated Antigen (TAA)” in its broadest sense relates to factors, which are primarily, if not exclusively expressed in tumors and thus can act as potential immune-therapeutic targets for antibody-based therapy. The primary and preferably exclusive expression of TAAs in tumor tissue ensures that the therapeutic antibody mediated immune reaction will be localized to the tumor only so that the above-described adverse events and effects on pharmacokinetic behavior are observed at least not to the same extent as for therapeutic antibodies which target antigens that are expressed both in tumor and normal tissues.

It is to be understood that expression of such TAAs must be seen before the background of accessibility of such expressed TAAs to antibodies and/or accessibility of such expressed TAAs to the immune system.

Thus, expression of TAAs may occur on the DNA or RNA level in normal tissue, which, however, does not translate into expression on the protein level. As a consequence such a TAA will not be expressed in normal tissues in an extent that would make it principally available for therapeutic antibodies as such antibodies are commonly understood to recognized antigens and/or epitopes involving stretches of amino acids.

Further, there may be tissues such as testis which are not functionally accessible to the immune system, e.g. in the sense that they do not show MHC expression and therefore cannot be targeted by T-cells, and which therefore are commonly considered to be immune privileged. Even if a TAA is expressed in such immune privileged normal tissue, an antibody binding to such a TAA would thus not trigger an immune response in such normal tissue. Again the immune response would be limited to the tumor tissue expressing the TAA.

A preferred group of TAAs are the so-called “cancer/testis antigens (CT-antigens)”. This group has emerged as a unique class of TAAs, which are expressed either in diverse tumors or normally in testis, i.e. an immune privileged tissue. An overview on the properties of CT-antigens including information on their genomic coding, function, tumor expression etc. can be found inter alia in Caballero et al., 2009, Cancer Science, 100(11), 2014-2021, the disclosure of which is incorporated by reference particularly with respect to the nature of CT antigens as well as the occurrence and distribution of specific CT antigens within different types of tumors (see e.g. Table 1 of Caballero et al., vide supra).

Detailed information about CT-antigens can be found in www.cta.lncc.br/. The information provided by this database, in particular with respect to gene families of CT-Antigens, specific family members, their chromosomal localization, CT identifiers and protein expression patterns in tumors are incorporated by reference.

Examples of CT-Antigens can be found in Table 1.

TABLE 1
List of preferred CT-antigens

| Gene family | CT-Antigen | CT-Identifier |
|---|---|---|
| MAGEA | MAGEA1 | CT1.1 |
| MAGEA | MAGEA2 | CT1.2 |
| MAGEA | MAGEA3 | CT1.3 |
| MAGEA | MAGEA4 | CT1.4 |
| MAGEA | MAGEA5 | CT1.5 |
| MAGEA | MAGEA6 | CT1.6 |
| MAGEA | MAGEA10 | CT1.10 |
| BAGE | BAGE | CT2.1 |
| GAGE | GAGE1 | CT4.1 |
| SSX | SSX1 | CT5.1 |
| SSX | SSX2 | CT5.2a |
| SSX | SSX4 | CT5.4 |
| NY-ESO-1 | CTAG1B | CT6.1 |
| NY-ESO-1 | CTAG1A | |
| NY-ESO-1 | CTAG2 | CT6.2a |
| NY-ESO-1 | LAGE-1b | CT6.2b |
| MAGEC1 | MAGEC1 | CT7.1 |
| MAGEC1 | MAGEC3 | CT7.2 |
| MAGEC2 | MAGEC2 | CT10 |
| XAGE | XAGE1 | CT12.1a |
| XAGE | XAGE2 | CT12.2 |
| CT47 | CT47A1 | CT47.1 |
| PRAME | PRAME | CT130 |

The term “CT-Antigen” is used interchangeably both for the gene family as well as for individual members of a gene family.

It is to be understood that if in the following reference is made to MAGEA3 binding antibodies or binding fragments thereof, this means that such antibodies bind to the CT-antigen MAGEA3. Such reference shall always include MAGEA3 binding antibodies or fragments thereof as mentioned hereinafter and in particular the specific antibodies and their sequence homologues as they are mentioned herein such as 122G3, 32H2, 34G9 or 102G10. The term MAGEA3 preferably refers to the human MAGEA3 protein and may thus designate a protein comprising an amino acid sequence of SEQ ID No:41.

If it is stated that an antibody or fragment thereof binds to MAGEA3, this means that the antibody or binding fragments thereof preferably binds specifically to said antigen, i.e. binds the antigen with greater affinity than other antigens. This particularly means that no other CT-antigens from different gene families such as NY-ESO are recognized by said antibody or binding fragment thereof.

For example, an antibody or fragment is specific for its cognate antigen when the variable regions of the antibody or fragment recognize and bind the cognate antigen with a detectable preference distinguishing the antigen from other known polypeptides of similar but not identical sequence by virtue of measurable differences in binding affinity. It will be understood that antigen-specific antibodies and fragments may also interact with other proteins (for example, S. aureus protein A or other antibodies in ELISA techniques) through interactions with sequences outside the variable region of the antibodies, and in particular, in the constant region of the antibody or fragment. It is to be understood that interaction of the MAGEA3 binding antibodies or binding fragments thereof with Protein A or G is not considered as an antigen-specific interaction. Screening assays to determine binding specificity of an antibody are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. As the MAGEA3 contemplated in the context of the present invention are typically only expressed in tumor tissue or immune privileged tissue, specific binding antibodies or fragments thereof will preferably detectably bind (as judged by common assays) in tumor tissue to MAGEA3 only, but not to other polypeptides which are expressed both in tumor tissue and normal tissue.

However, MAGEA3 is a member of the MAGEA family and shares significant homology with at least some of the other members of the MAGEA family such as MAGEA6 (about 96% homology) or MAGEA2 (about 84% homology). The homology with other family members such as MAGEA1 and MAGEA4 (about 68% homology) or MAGEA10 (about 50% homology) is lower.

While MAGEA3 binding antibodies or binding fragments thereof will usually be specific in the sense that they bind MAGEA3 stronger than e.g. other members of the MAGEA family, the MAGEA3 binding antibodies or binding fragments thereof may also bind to MAGEA6 and in particular human MAGEA6 given that it shares 96% sequence identity with MAGEA3. A MAGEA3 binding antibody or binding fragment as described herein may therefore also be designated as a MAGEA6 binding antibody or binding fragment thereof. Given the high homology between MAGEA3 and MAGEA6, it is assumed that these proteins have similar if not identical roles e.g. in cancer development so that such a pan reactivity towards MAGEA3 and MAGEA6 may be of therapeutic advantage. The capability of MAGEA3 binding antibodies or binding fragments thereof in accordance with the invention to bind also to MAGEA6 does not preclude that these antibodies will bind stronger to MAGEA3 than MAGEA6. In some embodiments, MAGEA3 binding antibodies or binding fragments thereof may optionally not even bind to MAGEA6.

It has been observed that the expression pattern of MAGEA3 and other family members such as MAGEA1, MAGEA4 and MAGEA10 can differ between cancer types, which may point to different roles of these proteins in cancer development and progression. In order to ensure that a pharmaceutically active MAGEA3 binding antibody or binding fragment thereof indeed only interferes with the function of MAGEA3, it can be preferred that the antibodies or binding fragments in accordance with the invention recognize only MAGEA3, but preferably not MAGEA4, MAGEA1 and/or MAGEA10. Such antibodies are considered to preferentially bind MAGEA3. It is to be understood that the statement that a MAGEA3 binding antibody or binding fragment thereof does not bind e.g. MAGEA4 is based on experiments that are commonly used to determine the preference of antibody binding towards certain targets. To this extent, one may use for example ELISA assays where different antigens such as MAGEA3, MAGEA4 etc. are coated on a substrate and subsequently binding of a particular antibody is tested. MAGEA3 binding antibodies and binding fragments thereof in accordance with the invention thus may not detectably bind to MAGEA1, MAGEA4 and MAGE10 over a negative control antibody as can be determined e.g. in common ELISA or Western Blot assays.

In some embodiments the present invention thus relates to MAGEA3 binding antibodies or binding fragments thereof which preferentially bind to MAGEA3 and thus do not bind to MAGEA4, MAGEA1, MAGEA10 and/or optionally even MAGEA2.

Antibodies or binding fragments thereof, regardless of whether they are MAGEA3 binding antibodies or binding fragments thereof or e.g. the other antibodies described herein such as the anti-CD40 agonistic antibodies may have an equilibrium dissociation constant (K_{D}) for the binding of the antibody (or the binding fragment thereof) to its antigen in the low nanomolar to low picomolar or even in the subpicomolar range (avidity). Thus the K_{D} may be in the range of about 0.1*10⁻¹² to about 1*10⁻⁸, preferably in the range of about 0.1*10⁻¹² to about 0.1*10⁻⁷, more preferably in the range of about 0.1*10⁻¹² to about 10*10⁻⁹, even more preferably in the range of about 0.1*10⁻¹² to about 1*10⁻⁹. The most preferred KDs may be in the range of about 0.1*10⁻¹² to about 0.1*10⁻⁹, in the range of about 0.1*10⁻¹² to about 10*10⁻¹² or in the range of about 0.1*10⁻¹² to about 1*10⁻¹² such as about 0.9*10⁻¹², about 0.8*10⁻¹², about 0.7*10⁻¹², about 0.6*10⁻¹² or about 0.5*10⁻¹². Thus MAGEA3 binding antibodies or binding fragments thereof as described hereinafter may have a K_{D} of about 300 pM or less, about 200 pM or less, about 100 pM or less, about 90 pM or less, about 80 pM or less, about 70 pM or less, about 60 pM or less, about 50 pM or less, about 40 pM or less, about 30 pM or less, or about 20 pM or less. Even lower K_{D}s may be achievable by optimization of CDRs.

The K_{D} is usually considered to be a measure for the affinity of an interaction between two molecules. Strictly speaking, affinity describes the strength of binding of a molecule to another molecule at a single site. However, an antibody usually has two binding sites for an antigen. The strength of this interaction is usually considered to be the avidity.

In the context of the present invention, the term “affinity” is used to describe both the strength of the interaction of e.g. a monovalent scFv to its antigen as well as the binding of a typical divalent antibody to its antigen.

K_{D} values and thus the affinity/avidity of the antibodies or binding fragments thereof can be determined making use of established approaches in the art.

Another measure of the affinity of an antibody such as the MAGEA3 binding antibodies or binding fragments described herein towards their antigen is the EC₅₀ concentration. Antibodies or binding fragments thereof, regardless of whether they are MAGEA3 binding antibodies or binding fragments thereof or e.g. the other antibodies described herein such as the anti-CD40 agonistic antibodies may have an EC₅₀ for the binding of the antibody (or the binding fragment thereof) to its antigen in the low nanomolar to low picomolar or even in the subpicomolar range. Thus the EC₅₀ may be in the range of about 0.1*10⁻¹² to about 1*10⁻⁸, preferably in the range of about 0.1*10⁻¹² to about 0.1*10⁻⁷, more preferably in the range of about 0.1*10⁻¹² to about 10*10⁻⁹, even more preferably in the range of about 0.1*10⁻¹² to about 1*10⁻⁹. The most preferred EC₅₀s may be in the range of about 0.1*10⁻¹² to about 0.1*10⁻⁹, in the range of about 0.1*10⁻¹² to about 10*10⁻¹² or in the range of about 0.1*10⁻¹² to about 1*10⁻¹² such as about 0.9*10⁻¹², about 0.8*10⁻¹², about 0.7*10⁻¹², about 0.6*10⁻¹² or about 0.5*10⁻¹². Thus MAGEA3 binding antibodies or binding fragments thereof as described hereinafter may have an EC₅₀ of about 300 pM or less, about 200 pM or less, about 100 pM or less, about 90 pM or less, about 80 pM or less, about 70 pM or less, about 60 pM or less, about 50 pM or less, about 40 pM or less, about 30 pM or less, or about 20 pM or less. Even lower EC₅₀s may be achievable by optimization of CDRs.

The EC₅₀ is determined as the concentration at which half-maximal binding of the antibody to its antigen in ELISA was observed.

The antibodies and binding fragments thereof as they are used in the context of the present invention, i.e. regardless of whether they are MAGEA3 binding antibodies or binding fragments thereof or e.g. the other antibodies described herein such as the anti-CD40 agonistic antibodies as mentioned hereinafter may be preferably monoclonal chimeric, humanized or human antibodies. These antibodies are preferably of the IgG class.

At least for the MAGEA3 binding antibodies or binding fragments thereof it can be preferred to use monoclonal human antibodies. Such antibodies are preferably “patient-derived”.

A “patient-derived” human monoclonal antibody refers to an antibody which has been obtained from a patient suffering from a tumor expressing a TAA as defined above and in particular MAGEA3 and/or MAGEA6. In some embodiments, it can be preferred to isolate such antibodies from patients which suffer from a tumor expressing a TAA as defined above and in particular MAGEA3 and/or MAGEA6 and display a favorable clinical course of disease. Such favorable clinical course of disease may become apparent e.g. from quality of life, overall survival, improved time to progression and/or improved RECIST criteria. RECIST (“Response Evaluation Criteria In Solid Tumors”) is e.g. used to determine whether a patient has shown a complete or at least partial response to treatment of such tumor. An explanation and overview of these criteria can be found inter alia at Eisenhauer et al., (2009) European Journal of Cancer, 228-247 or at www.eortc.be/recist/ and are incorporated by reference.

It is to be understood that a favorable clinical course of disease may be observed in patients which have been diagnosed with a tumor and which e.g. have received non-specific chemotherapy and/or vaccination with a CT antigen such as MAGEA3. A patient which has shown a favorable clinical course of disease, may be eligible for isolation and identification of MAGEA3 binding antibodies even if the patient which has been diagnosed with a tumor, has not been e.g. vaccinated with a MAGEA3 antigen.

The use of such patient-derived antibodies is assumed to provide for at least comparable efficacy even if they are administered to patients different from the ones from which they have been isolated. For example, the specific MAGEA3 binding antibodies or binding fragments thereof mentioned herein have been isolated from patients, which suffered from melanoma or breast cancer and which had been vaccinated with full length human MAGEA3. Vaccination with MAGEA3 may be supported with adjuvants including immunostimulants such CpG. Such antibodies are assumed to be able to recruit CD4⁺, CD8⁺ cytotoxic T cells into xenografted tumors of mice and also to tumors of patients which express MAGEA3 and/or MAGEA6.

A patient-derived human monoclonal antibody can be assumed to show efficacy also in other human patients which suffer from a hyperproliferative disease such as a cancer being characterized by MAGEA3 and/or MAGEA6 overexpression given that it has been isolated from a patient which has shown a favorable clinical course of disease as mentioned above. These antibodies have a fully human sequence and thus should pose no problem e.g. with respect to immunogenicity.

Some examples of such patient derived human monoclonal MAGEA3 binding antibodies include 122G3, 32H2, 34G9 or 102G10. Of these antibodies, which have been obtained from MAGEA3 vaccinated patients, 32H2 and 34G9 were obtained from patients receiving in addition the adjuvant CpG.

The variable heavy chain of 122G3 is e.g. encoded by SEQ ID No. 1. The variable light chain of 122G3 is e.g. encoded by SEQ ID No. 2. The variable heavy chain of 122G3 thus has an amino acid sequence of SEQ ID No. 3. The variable light chain of 122G3 thus has an amino acid sequence of SEQ ID No. 4. As regards the variable heavy chain of 122G3, the CDR1 has an amino acid sequence of SEQ ID No. 5, the CDR2 has an amino acid sequence of SEQ ID No. 6 and the CDR3 has an amino acid sequence of SEQ ID No. 7. As regards the variable light chain of 122G3, the CDR1 has an amino acid sequence of SEQ ID No. 8, the CDR2 has an amino acid sequence of SEQ ID No. 9 and the CDR3 has an amino acid sequence of SEQ ID No. 10.

The variable heavy chain of 32H2 is e.g. encoded by SEQ ID No. 11. The variable light chain of 32H2 is e.g. encoded by SEQ ID No. 12. The variable heavy chain of 32H2 thus has an amino acid sequence of SEQ ID No. 13. The variable light chain of 32H2 thus has an amino acid sequence of SEQ ID No. 14. As regards the variable heavy chain of 32H2, the CDR1 has an amino acid sequence of SEQ ID No. 15, the CDR2 has an amino acid sequence of SEQ ID No. 16 and the CDR3 has an amino acid sequence of SEQ ID No. 17. As regards the variable light chain of 32H2, the CDR1 has an amino acid sequence of SEQ ID No. 18, the CDR2 has an amino acid sequence of SEQ ID No. 19 and the CDR3 has an amino acid sequence of SEQ ID No. 20.

The variable heavy chain of 34G9 is e.g. encoded by SEQ ID No. 21. The variable light chain of 34G9 is e.g. encoded by SEQ ID No. 22. The variable heavy chain of 34G9 thus has an amino acid sequence of SEQ ID No. 23. The variable light chain of 34G9 thus has an amino acid sequence of SEQ ID No. 24. As regards the variable heavy chain of 34G9, the CDR1 has an amino acid sequence of SEQ ID No. 25, the CDR2 has an amino acid sequence of SEQ ID No. 26 and the CDR3 has an amino acid sequence of SEQ ID No. 27. As regards the variable light chain of 34G9, the CDR1 has an amino acid sequence of SEQ ID No. 28, the CDR2 has an amino acid sequence of SEQ ID No. 29 and the CDR3 has an amino acid sequence of SEQ ID No. 30.

The variable heavy chain of 102G10 is e.g. encoded by SEQ ID No. 31. The variable light chain of 102G10 is e.g. encoded by SEQ ID No. 32. The variable heavy chain of 102G10 thus has an amino acid sequence of SEQ ID No. 33. The variable light chain of 102G10 thus has an amino acid sequence of SEQ ID No. 34. As regards the variable heavy chain of 102G10, the CDR1 has an amino acid sequence of SEQ ID No. 35, the CDR2 has an amino acid sequence of SEQ ID No. 36 and the CDR3 has an amino acid sequence of SEQ ID No. 37. As regards the variable light chain of 102G10, the CDR1 has an amino acid sequence of SEQ ID No. 38, the CDR2 has an amino acid sequence of SEQ ID No. 39 and the CDR3 has an amino acid sequence of SEQ ID No. 40.

As the specificity and affinity of an antibody is largely determined by its variable heavy chain and light chain sequences and in particular by the CDRs thereof, it can be assumed that these properties are maintained even if the variable heavy chain and light chain sequences or CDRs thereof of human, patient-derived antibodies are transferred into other backbones. The present invention therefore also relates to MAGE3 binding antibodies and binding fragments thereof, which make use of variable heavy chain and light chain sequences and in particular by the CDRs thereof derived from human patient-derived antibodies, which are e.g. monoclonal chimeric or humanized antibodies. It is in fact known that introducing e.g. mouse-derived amino acids in framework positions of otherwise fully human antibodies may e.g. improve the ADCC response. Rules for selecting such modifications may e.g. be taken from e.g. EP 0 451 216. It is also known that, once a CDR or set of CDRs has been identified, it is possible to perform e.g. conservative amino acid substitutions in the CDR(s), while maintaining the affinity and/or specificity of an antibody. Such substitutions, which preferably are conservative, i.e. replacement by an amino acid with another amino acid with comparable physico-chemical properties (e.g. Ala by Leu, or Glu by Asp), but do not necessarily have to be conservative, can be identified by changing amino acids in the CDRs and/or framework regions and then testing the modified antibody vs. the original antibody for its antigen binding using e.g. BIACORE measurements.

Examples of MAGEA3 binding antibodies or binding fragments thereof therefore include monoclonal antibodies or binding fragments thereof comprising a light chain variable region and/or a heavy chain variable region, wherein

a) the light chain variable region comprises at least a CDR1 selected from SEQ ID Nos.: 8, 18, 28, 38 or sequences at least 80% identical thereto, a CDR2 selected from SEQ ID Nos.: 9, 19, 29, 39 or sequences at least 80% identical thereto, and/or a CDR3 selected from SEQ ID Nos.: 10, 20, 30, 40 or sequences at least 80% identical thereto; and/or wherein

b) the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and/or a CDR3 selected from SEQ ID Nos.: 7, 17, 27, 37 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 8 or sequences at least 80% identical thereto, a CDR2 selected from SEQ ID Nos.: 9 or sequences at least 80% identical thereto, and/or a CDR3 selected from SEQ ID Nos.: 10 or sequences at least 80% identical thereto; and/or wherein

b) the heavy chain variable region comprises at least a CDR1 selected from SEQ ID Nos.: 5 or sequences at least 80% identical thereto, a CDR2 selected from SEQ ID Nos.: 6 or sequences at least 80% identical thereto, and/or a CDR3 selected from SEQ ID Nos.: 7 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 18 or sequences at least 80% identical thereto, a CDR2 selected from SEQ ID Nos.: 19 or sequences at least 80% identical thereto, and/or a CDR3 selected from SEQ ID Nos.: 20 or sequences at least 80% identical thereto; and/or wherein

b) the heavy chain variable region comprises at least a CDR1 selected from SEQ ID Nos.: 15 or sequences at least 80% identical thereto, a CDR2 selected from SEQ ID Nos.: 16 or sequences at least 80% identical thereto, and/or a CDR3 selected from SEQ ID Nos.: 17 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 28 or sequences at least 80% identical thereto, a CDR2 selected from SEQ ID Nos.: 29 or sequences at least 80% identical thereto, and/or a CDR3 selected from SEQ ID Nos.: 30 or sequences at least 80% identical thereto; and/or wherein

b) the heavy chain variable region comprises at least a CDR1 selected from SEQ ID Nos.: 25 or sequences at least 80% identical thereto, a CDR2 selected from SEQ ID Nos.: 26 or sequences at least 80% identical thereto, and/or a CDR3 selected from SEQ ID Nos.: 27 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 38 or sequences at least 80% identical thereto, a CDR2 selected from SEQ ID Nos.: 39 or sequences at least 80% identical thereto, and/or a CDR3 selected from SEQ ID Nos.: 40 or sequences at least 80% identical thereto; and/or wherein

b) the heavy chain variable region comprises at least a CDR1 selected from SEQ ID Nos.: 35 or sequences at least 80% identical thereto, a CDR2 selected from SEQ ID Nos.: 36 or sequences at least 80% identical thereto, and/or a CDR3 selected from SEQ ID Nos.: 37 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 8, 18, 28, 38 or sequences at least 80% identical thereto, a CDR2 selected from SEQ ID Nos.: 9, 19, 29, 39 or sequences at least 80% identical thereto, and a CDR3 selected from SEQ ID Nos.: 10, 20, 30, 40 or sequences at least 80% identical thereto; and/or wherein

b) the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and a CDR3 selected from SEQ ID Nos.: 7, 17, 27, 37 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 8 or sequences at least 80% identical thereto, a CDR2 selected from SEQ ID Nos.: 9 or sequences at least 80% identical thereto, and a CDR3 selected from SEQ ID Nos.: 10 or sequences at least 80% identical thereto; and/or wherein

b) the heavy chain variable region comprises at least a CDR1 selected from SEQ ID Nos.: 5 or sequences at least 80% identical thereto, a CDR2 selected from SEQ ID Nos.: 6 or sequences at least 80% identical thereto, and a CDR3 selected from SEQ ID Nos.: 7 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 18 or sequences at least 80% identical thereto, a CDR2 selected from SEQ ID Nos.: 19 or sequences at least 80% identical thereto, and a CDR3 selected from SEQ ID Nos.: 20 or sequences at least 80% identical thereto; and/or wherein

b) the heavy chain variable region comprises at least a CDR1 selected from SEQ ID Nos.: 15 or sequences at least 80% identical thereto, a CDR2 selected from SEQ ID Nos.: 16 or sequences at least 80% identical thereto, and a CDR3 selected from SEQ ID Nos.: 17 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 28 or sequences at least 80% identical thereto, a CDR2 selected from SEQ ID Nos.: 29 or sequences at least 80% identical thereto, and a CDR3 selected from SEQ ID Nos.: 30 or sequences at least 80% identical thereto; and/or wherein

b) the heavy chain variable region comprises at least a CDR1 selected from SEQ ID Nos.: 25 or sequences at least 80% identical thereto, a CDR2 selected from SEQ ID Nos.: 26 or sequences at least 80% identical thereto, and a CDR3 selected from SEQ ID Nos.: 27 or sequences at least 80% identical thereto.

Other examples of MAGEA3 binding antibodies or binding fragments thereof include monoclonal antibodies or binding fragments thereof comprising a light chain variable region and/or a heavy chain variable region, wherein

a) the light chain variable region comprises at least a CDR1 selected from SEQ ID Nos.: 38 or sequences at least 80% identical thereto, a CDR2 selected from SEQ ID Nos.: 39 or sequences at least 80% identical thereto, and a CDR3 selected from SEQ ID Nos.: 40 or sequences at least 80% identical thereto; and/or wherein

b) the heavy chain variable region comprises at least a CDR1 selected from SEQ ID Nos.: 35 or sequences at least 80% identical thereto, a CDR2 selected from SEQ ID Nos.: 36 or sequences at least 80% identical thereto, and a CDR3 selected from SEQ ID Nos.: 37 or sequences at least 80% identical thereto.

Preferably, in all these embodiments the sequence identity is at least about 85%, more preferably at least about 90%, even more preferably at least about 95%, and most preferably at least about 96%, 97%, 98% or about 99%. Sequence identity may be determined over the whole length of the respective sequences.

The determination of percent identity between two sequences is preferably accomplished using the mathematical algorithm of Karlin and Altschul (1993) Proc. Natl. Acad. Sci. USA 90: 5873-5877. Such an algorithm is incorporated into the BLASTn and BLASTp programs of Altschul et al. (1990) J. Mol. Biol. 215: 403-410 available at NCBI (www.ncbi.nlm.nih.gov/blast/Blast.cge). The determination of percent identity is performed with the standard parameters of the BLASTn and BLASTp programs.

BLAST polynucleotide searches are performed with the BLASTn program. For the general parameters, the “Max Target Sequences” box may be set to 100, the “Short queries” box may be ticked, the “Expect threshold” box may be set to 10 and the “Word Size” box may be set to 28. For the scoring parameters the “Match/mismatch Scores” may be set to 1,-2 and the “Gap Costs” box may be set to linear. For the Filters and Masking parameters, the “Low complexity regions” box may not be ticked, the “Species-specific repeats” box may not be ticked, the “Mask for lookup table only” box may be ticked, and the “Mask lower case letters” box may not be ticked.

BLAST protein searches are performed with the BLASTp program. For the general parameters, the “Max Target Sequences” box may be set to 100, the “Short queries” box may be ticked, the “Expect threshold” box may be set to 10 and the “Word Size” box may be set to “3”. For the scoring parameters the “Matrix” box may be set to “BLOSUM62”, the “Gap Costs” Box may be set to “Existence: 11 Extension:1”, the “Compositional adjustments” box may be set to “Conditional compositional score matrix adjustment”. For the Filters and Masking parameters the “Low complexity regions” box may not be ticked, the “Mask for lookup table only” box may not be ticked and the “Mask lower case letters” box may not be ticked.

The above-mentioned CDRs of a light and heavy chain variable region are preferably embedded in the framework and constant region of a human-derived antibody, i.e. in the sequences as determined for antibodies obtained from human patients as described herein. Preferably these antibodies are of the IgG class.

However, the above-mentioned CDRs of a light and heavy chain variable region may also be embedded in human sequences of framework and constant regions derived from other human antibodies, particularly if such sequences have been shown to be effective in antibody dependent cell mediated cytotoxicity (ADCC). In this context, one may e.g. use the human constant and framework sequences of humanized therapeutic antibodies that have been successfully used for therapeutic applications. The above-mentioned CDRs of a light and heavy chain variable region are preferably incorporated into the framework and constant regions of such humanized antibodies of the human IgG class.

Further, the above-mentioned CDRs of a light and heavy chain variable region may be embedded in essentially human sequences for framework and constant regions. However, particularly the framework regions, but also the constant regions may comprise amino acids as they are e.g. typically found in mouse antibodies which are known to enhance antigen binding and/or e.g. ADCC (see e.g. European patent application EP 0 451 216). Preferably these antibodies are of the IgG class.

Other MAGEA3 binding antibodies and binding fragments relate to antibodies or binding fragments thereof comprising a light chain variable region comprising SEQ ID Nos.: 4, 14, 24, 34 or sequences at least 80% identical thereto and/or a heavy chain variable region comprising SEQ ID Nos.: 3, 13, 23, 33 or sequences at least 80% identical thereto.

Other MAGEA3 binding antibodies and binding fragments relate to antibodies or binding fragments thereof comprising a light chain variable region comprising SEQ ID Nos.: 4, 14, 24, 34 or sequences at least 80% identical thereto and a heavy chain variable region comprising SEQ ID Nos.: 3, 13, 23, 33 or sequences at least 80% identical thereto.

Other MAGEA3 binding antibodies and binding fragments relate to antibodies or binding fragments thereof comprising a light chain variable region comprising SEQ ID No.: 4 or sequences at least 80% identical thereto and a heavy chain variable region comprising SEQ ID Nos.: 3, 13, 23, 33 or sequences at least 80% identical thereto.

Other MAGEA3 binding antibodies and binding fragments relate to antibodies or binding fragments thereof comprising a light chain variable region comprising SEQ ID No.: 14 or sequences at least 80% identical thereto and a heavy chain variable region comprising SEQ ID Nos.: 3, 13, 23, 33 or sequences at least 80% identical thereto.

Other MAGEA3 binding antibodies and binding fragments relate to antibodies or binding fragments thereof comprising a light chain variable region comprising SEQ ID No.: 24 or sequences at least 80% identical thereto and a heavy chain variable region comprising SEQ ID Nos.: 3, 13, 23, 33 or sequences at least 80% identical thereto.

Other MAGEA3 binding antibodies and binding fragments relate to antibodies or binding fragments thereof comprising a light chain variable region comprising SEQ ID No.: 34 or sequences at least 80% identical thereto and a heavy chain variable region comprising SEQ ID Nos.: 3, 13, 23, 33 or sequences at least 80% identical thereto.

Other MAGEA3 binding antibodies and binding fragments relate to antibodies or binding fragments thereof comprising a light chain variable region comprising SEQ ID Nos.: 4, 14, 24, 34 or sequences at least 80% identical thereto and a heavy chain variable region comprising SEQ ID Nos.: 3 or sequences at least 80% identical thereto.

Other MAGEA3 binding antibodies and binding fragments relate to antibodies or binding fragments thereof comprising a light chain variable region comprising SEQ ID Nos.: 4, 14, 24, 34 or sequences at least 80% identical thereto and a heavy chain variable region comprising SEQ ID Nos.: 13 or sequences at least 80% identical thereto.

Other MAGEA3 binding antibodies and binding fragments relate to antibodies or binding fragments thereof comprising a light chain variable region comprising SEQ ID Nos.: 4, 14, 24, 34 or sequences at least 80% identical thereto and a heavy chain variable region comprising SEQ ID Nos.: 23 or sequences at least 80% identical thereto.

Other MAGEA3 binding antibodies and binding fragments relate to antibodies or binding fragments thereof comprising a light chain variable region comprising SEQ ID Nos.: 4, 14, 24, 34 or sequences at least 80% identical thereto and a heavy chain variable region comprising SEQ ID Nos.: 33 or sequences at least 80% identical thereto.

Other MAGEA3 binding antibodies and binding fragments relate to antibodies or binding fragments thereof comprising a light chain variable region comprising SEQ ID Nos.: 4 or sequences at least 80% identical thereto and a heavy chain variable region comprising SEQ ID Nos.: 3 or sequences at least 80% identical thereto.

Other MAGEA3 binding antibodies and binding fragments relate to antibodies or binding fragments thereof comprising a light chain variable region comprising SEQ ID Nos.: 14 or sequences at least 80% identical thereto and a heavy chain variable region comprising SEQ ID Nos.: 13 or sequences at least 80% identical thereto.

Other MAGEA3 binding antibodies and binding fragments relate to antibodies or binding fragments thereof comprising a light chain variable region comprising SEQ ID Nos.: 24 or sequences at least 80% identical thereto and a heavy chain variable region comprising SEQ ID Nos.: 23 or sequences at least 80% identical thereto.

Other MAGEA3 binding antibodies and binding fragments relate to antibodies or binding fragments thereof comprising a light chain variable region comprising SEQ ID Nos.: 34 or sequences at least 80% identical thereto and a heavy chain variable region comprising SEQ ID Nos.: 33 or sequences at least 80% identical thereto.

Preferably, in all these embodiments the sequence identity is at least about 85%, more preferably at least about 90%, even more preferably at least about 95% and most preferably at least about 98% or at least about 99%. Sequence identity is determined as described above. Sequence identity may be determined over the whole length of the respective sequence.

The above-mentioned light and heavy chain variable regions are preferably embedded in the constant regions of a human-derived antibody, i.e. in the sequences as determined for antibodies obtained from human patients as described herein. Preferably these antibodies are of the IgG class such as the IgG1 class.

However, the above-mentioned light and heavy chain variable regions may also be embedded in human sequences of constant regions derived from other human antibodies, particularly if such sequences have been shown to be effective in ADCC. In this context, one may e.g. use the human constant sequences of humanized therapeutic antibodies that have been successfully used for therapeutic applications. The above-mentioned light and heavy chain variable regions are preferably incorporated into the constant regions of such humanized antibodies of the human IgG class.

Further, the above-mentioned light and heavy chain variable regions may be embedded in essentially human sequences for constant regions. However, the constant regions may comprise amino acids as they are e.g. typically found in mouse antibodies, which are known to enhance ADCC. Preferably these antibodies are of the IgG class.

The MAGEA3 binding antibodies or binding fragments in accordance with the invention may bind to epitope(s) comprised within SEQ ID No. 42, SEQ ID No. 43 and/or SEQ ID No. 44.

Preferably, the MAGEA3 binding antibodies or binding fragments in accordance with the invention may bind to epitope(s) comprised within SEQ ID No. 45, SEQ ID No. 46, SEQ ID No. 47 and/or SEQ ID No. 48.

Also preferably, the MAGEA3 binding antibodies or binding fragments in accordance with the invention may bind to epitope(s) comprised within SEQ ID No. 108.

The invention also contemplates using MAGEA3 binding antibodies and binding fragments thereof binding substantially to the same epitope or parts of the same epitope as do the MAGEA3 binding antibodies and binding fragments as described above

Further, the invention considers using MAGEA3 binding antibodies and binding fragments thereof competing with MAGEA3 binding antibodies and binding fragments thereof as described above for binding to MAGEA3 and preferably for binding to human MAGEA3.

Epitope mapping may be undertaken by producing different fragments of MAGEA3 and to then test these fragments for binding to antibodies or the binding fragments thereof. Binding may be measured using ELISA. Binding may also be determined using Biacore®. One may also use commercially available peptide arrays such as PepSpot™ from JPT Peptide Technologies GmbH (Berlin, Germany), solutions offered by Peptides&Elephants, Nuthetal, Germany or proteomics-based mass spectrometry methods. Competition for binding to a particular antigen or epitope can be determined using assays known in the art. For example one may label an antibody in accordance with the invention and test for its binding to MAGEA3. Subsequently, one adds unlabeled 122G3 (or any other MAGEA3 binding antibody) and determines whether it affects binding of the labeled antibody, or binding of the labeled antibody is studied in presence or absence of various concentrations of such unlabeled MAGEA3 binding antibody. Such label could be radioactive or fluorescent or other kinds of detectable label.

Competition for binding to a particular antigen or epitope is determined by a reduction in binding to antigen or epitope of at least about 50%, or at least about 70%, or at least about 80%, or at least about 90%, or at least about 95%, or at least about 99% or about 100% for the antibody in accordance with the invention. Binding may be measured using Biacore® equipment, various fluorescence detection technologies (e.g. Fluorescence correlation spectroscopy, fluorescence cross-correlation, Fluorescence Lifetime measurements etc.) or various types of radioimmunoassays or other assays used to follow antibody binding to a target molecule.

As mentioned above, the present invention considers MAGEA3 binding antibodies or binding fragments thereof. A full-length antibody includes a constant domain and a variable domain. The constant region need not be present in an antigen-binding fragment of an antibody.

Binding fragments may thus include portions of an intact full-length antibody, such as an antigen binding or variable region of the complete antibody. Examples of antibody fragments include Fab, F(ab′)₂, Id and Fv fragments; diabodies; linear antibodies; single-chain antibody molecules (e.g., scFv); multispecific antibody fragments such as bispecific, trispecific, and multispecific antibodies (e.g., diabodies, triabodies, tetrabodies); minibodies; chelating recombinant antibodies; tribodies or bibodies; intrabodies; nanobodies; small modular immunopharmaceuticals (SMIP), binding-domain immunoglobulin fusion proteins; camelized antibodies; VHH containing antibodies; and any other polypeptides formed from antibody fragments. The skilled person is aware that the antigen-binding function of an antibody can be performed by fragments of a full-length antibody.

A Fab fragment consists of the VL, VH, CL and CH1 domains. An F(ab′)² fragment comprises two Fab fragments linked by a disulfide bridge at the hinge region. An Fd is the VH and CH1 domains of a single arm of an antibody. An Fv fragment is the VL and VH domains of a single arm of an antibody.

Binding fragments also encompass monovalent or multivalent, or monomeric or multimeric (e.g. tetrameric), CDR-derived binding domains.

The MAGEA3 binding antibodies and binding fragments thereof may also encompass variants of the exemplary antibodies, binding fragments and sequences disclosed herein. Variants include peptides and polypeptides comprising one or more amino acid sequence substitutions, deletions, and/or additions that have the same or substantially the same affinity and specificity of epitope binding as one or more of the exemplary antibodies, fragments and sequences disclosed herein. Thus, variants include peptides and polypeptides comprising one or more amino acid sequence substitutions, deletions, and/or additions to the exemplary antibodies, fragments and sequences disclosed herein where such substitutions, deletions and/or additions do not cause substantial changes in affinity and specificity of epitope binding. For example, a variant of an antibody or fragment may result from one or more changes to an antibody or fragment comprising one or more of amino acid sequence of SEQ ID NOS: 3, 4 etc. or where the changed antibody or fragment has the same or substantially the same affinity and specificity of epitope binding as the starting sequence.

Antibodies or binding fragments thereof as far as they are generally referred to in the context of the present invention may also be part of larger immunoadhesion molecules, formed by covalent or non-covalent association of the antibody or antibody portion with e.g. one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibodies and fragments comprising immunoadhesion molecules can be obtained using standard recombinant DNA techniques, as described herein. Preferred antigen binding portions are complete domains or pairs of complete domains.

The binding antibodies and binding fragments of the present invention may also encompass domain antibody (dAb) fragments (Ward et al., Nature 341:544-546, 1989), which consist of a V_{H} domain. The antibodies and binding fragments of the present invention also encompass diabodies, which are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., EP 404,097; WO 93/11161; Holliger et al., Proc. Natl. Acad. Sci. USA 90:6444-6448, 1993, and Poljak et al., Structure 2:1121-1123, 1994). Diabodies can be bispecific or monospecific.

As mentioned the antibodies and binding fragments of the present invention also encompass single-chain antibody fragments (scFv). An scFv comprises an antibody heavy chain variable region (V_{H}) operably linked to an antibody light chain variable region (V_{L}) wherein the heavy chain variable region and the light chain variable region, together or individually, form a binding site. A scFv may comprise a V_{H} region at the amino-terminal end and a V_{L} region at the carboxy-terminal end. Alternatively, scFv may comprise a V_{L} region at the amino-terminal end and a V_{H} region at the carboxy-terminal end. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883).

A scFv may optionally further comprise a polypeptide linker between the heavy chain variable region and the light chain variable region. Such polypeptide linkers generally comprise between 1 and 50 amino acids, alternatively between 3 and 12 amino acids, alternatively 2 amino acids. An example of a linker peptide for linking heavy and light chains in a scFv comprises the 5 amino acid sequence Gly-Gly-Gly-Gly-Ser. Other examples comprise one or more tandem repeats of this sequence (for example, a polypeptide comprising two to four repeats of Gly-Gly-Gly-Gly-Ser) to create linkers.

The antibodies and binding fragments of the present invention also encompass heavy chain antibodies (HCAb). Exceptions to the H₂L₂ structure of conventional antibodies occur in some isotypes of the immunoglobulins found in camelids (camels, dromedaries and llamas; Hamers-Casterman et al., 1993 Nature 363: 446; Nguyen et al., 1998 J. Mol. Biol. 275: 413), wobbegong sharks (Nuttall et al., Mol Immunol. 38:313-26, 2001), nurse sharks (Greenberg et al., Nature 374:168-73, 1995; Roux et al., 1998 Proc. Nat. Acad. Sci. USA 95: 11804), and in the spotted ratfish (Nguyen, et al., “Heavy-chain antibodies in Camelidae; a case of evolutionary innovation,” 2002 Immunogenetics 54(1): 39-47). These antibodies can apparently form antigen-binding regions using only heavy chain variable region, in that these functional antibodies are dimers of heavy chains only (referred to as “heavy-chain antibodies” or “HCAbs”). Accordingly, some embodiments of the present antibodies and binding fragments may be heavy chain antibodies (HCAb) that specifically bind to the tumor-associated antigen. For example, heavy chain antibodies that are a class of IgG and devoid of light chains are produced by animals of the genus Camelidae that includes camels, dromedaries and llamas (Hamers-Casterman et al., Nature 363:446-448 (1993)). HCAbs have a molecular weight of about 95 kDa instead of the about 160 kDa molecular weight of conventional IgG antibodies. Their binding domains consist only of the heavy-chain variable domains, often referred to as V_{HH} to distinguish them from conventional V_{H}. Muyldermans et al., J. Mol. Recognit. 12:131-140 (1999). The variable domain of the heavy-chain antibodies is sometimes referred to as a nanobody (Cortez-Retamozo et al., Cancer Research 64:2853-57, 2004). A nanobody library may be generated from an immunized dromedary as described in Conrath et al., (Antimicrob Agents Chemother 45: 2807-12, 2001) or using recombinant methods.

Since the first constant domain (C_{H1}) is absent (spliced out during mRNA processing due to loss of a splice consensus signal), the variable domain (V_{HH}) is immediately followed by the hinge region, the C_{H2} and the C_{H3} domains (Nguyen et al., Mol. Immunol. 36:515-524 (1999); Woolven et al., Immunogenetics 50:98-101 (1999)). Camelid V_{HH} reportedly recombines with IgG2 and IgG3 constant regions that contain hinge, CH2, and CH3 domains and lack a CH1 domain (Hamers-Casterman et al., supra). For example, llama IgG1 is a conventional (H₂L₂) antibody isotype in which V_{H} recombines with a constant region that contains hinge, CH1, CH2 and CH3 domains, whereas the llama IgG2 and IgG3 are heavy chain-only isotypes that lack CH1 domains and that contain no light chains.

Although the HCAbs are devoid of light chains, they have an antigen-binding repertoire. The genetic generation mechanism of HCAbs is reviewed in Nguyen et al. Adv. Immunol 79:261-296 (2001) and Nguyen et al., Immunogenetics 54:39-47 (2002). Sharks, including the nurse shark, display similar antigen receptor-containing single monomeric V-domains. Irving et al., J. Immunol. Methods 248:31-45 (2001); Roux et al., Proc. Natl. Acad. Sci. USA 95:11804 (1998).

V_{HH}s comprise small intact antigen-binding fragments (for example, fragments that are about 15 kDa, 118-136 residues). Camelid V_{HH} domains have been found to bind to antigen with high affinity (Desmyter et al., J. Biol. Chem. 276:26285-90, 2001), with V_{HH} affinities typically in the nanomolar range and comparable with those of Fab and scFv fragments. V_{HH}s are highly soluble and more stable than the corresponding derivatives of scFv and Fab fragments. V_{H} fragments have been relatively difficult to produce in soluble form, but improvements in solubility and specific binding can be obtained when framework residues are altered to be more V_{HH}-like. (See, for example, Reichman et al., J Immunol Methods 1999, 231:25-38.) V_{HH}s carry amino acid substitutions that make them more hydrophilic and prevent prolonged interaction with BiP (Immunoglobulin heavy-chain binding protein), which normally binds to the H-chain in the Endoplasmic Reticulum (ER) during folding and assembly, until it is displaced by the L-chain. Because of the V_{HH}s' increased hydrophilicity, secretion from the ER is improved.

Functional V_{HH}s may be obtained by proteolytic cleavage of HCAb of an immunized camelid, by direct cloning of V_{HH} genes from B-cells of an immunized camelid resulting in recombinant V_{HH}s, or from naive or synthetic libraries. V_{HH}s with desired antigen specificity may also be obtained through phage display methodology. Using V_{HH}s in phage display is much simpler and more efficient compared to Fabs or scFvs, since only one domain needs to be cloned and expressed to obtain a functional antigen-binding fragment. Muyldermans, Biotechnol. 74:277-302 (2001); Ghahroudi et al., FEBS Lett. 414:521-526 (1997); and van der Linden et al., J. Biotechnol. 80:261-270 (2000). Methods for generating antibodies having camelid heavy chains are also described in U.S. Patent Publication Nos. 20050136049 and 20050037421.

The binding antibodies and binding fragments thereof may also encompass any of the e.g. foregoing specifically mentioned amino acid sequences of the light or heavy chains with one or more conservative substitutions (e.g., 1, 2, 3, 4, 5, 6, 7, 8, 9, 10, 11, 12, 13, 14, or 15 conservative substitutions). One can determine the positions of an amino acid sequence that are candidates for conservative substitutions, and one can select synthetic and naturally-occurring amino acids that effect conservative substitutions for any particular amino acids. Consideration for selecting conservative substitutions include the context in which any particular amino acid substitution is made, the hydrophobicity or polarity of the side-chain, the general size of the side chain, and the pK value of side-chains with acidic or basic character under physiological conditions. For example, lysine, arginine, and histidine are often suitably substituted for each other. As is known in the art, this is because all three amino acids have basic side chains, whereas the pK value for the side-chains of lysine and arginine are much closer to each other (about 10 and 12) than to histidine (about 6). Similarly, glycine, alanine, valine, leucine, and isoleucine are often suitably substituted for each other, with the proviso that glycine is frequently not suitably substituted for the other members of the group. Other groups of amino acids frequently suitably substituted for each other include, but are not limited to, the group consisting of glutamic and aspartic acids; the group consisting of phenylalanine, tyrosine, and tryptophan; and the group consisting of serine, threonine, and, optionally, tyrosine.

By making conservative modifications to the amino acid sequence or corresponding modifications to the encoding nucleotides, one can produce antibodies or binding fragments thereof having functional and chemical characteristics similar to those of the exemplary antibodies and fragments disclosed herein.

The binding antibodies and binding fragments thereof as they are mentioned in the context of the present invention may encompass derivatives of the exemplary antibodies, fragments and sequences disclosed herein. Derivatives include polypeptides or peptides, or variants, fragments or derivatives thereof, which have been chemically modified. Examples include covalent attachment of one or more polymers, such as water soluble polymers, N-linked, or O-linked carbohydrates, sugars, phosphates, and/or other such molecules such as detectable labels, such as fluorophores.

Labeling agents may be coupled either directly or indirectly to the antibodies or antigens of the invention. One example of indirect coupling is by use of a spacer moiety. Furthermore, the antibodies of the present invention can comprise a further domain, said domain being linked by covalent or noncovalent bonds. The linkage can be based on genetic fusion according to the methods known in the art and described above or can be performed by, e.g., chemical cross-linking as described in, e.g., international application WO 94/04686. The additional domain present in the fusion protein comprising the antibody of the invention may preferably be linked by a flexible linker, advantageously a polypeptide linker, wherein said polypeptide linker comprises plural, hydrophilic, peptide-bonded amino acids of a length sufficient to span the distance between the C-terminal end of said further domain and the N-terminal end of the antibody of the invention or vice versa. The therapeutically or diagnostically active agent can be coupled to the antibody of the invention or an antigen-binding fragment thereof by various means. This includes, for example, single-chain fusion proteins comprising the variable regions of the antibody of the invention coupled by covalent methods, such as peptide linkages, to the therapeutically or diagnostically active agent. Further examples include molecules, which comprise at least an antigen-binding fragment coupled to additional molecules covalently, or non-covalently include those in the following non-limiting illustrative list. Traunecker et al., Int. J. Cancer Surp. SuDP 7 (1992), 51-52, describes the bispecific reagent janusin in which the Fv region directed to CD3 is coupled to soluble CD4 or to other ligands such as OVCA and IL-7. Similarly an Fv region directed to MAGEA3 may be coupled to portions of e.g. an anti-CD40 agonistic antibody and/or portions of an anti-CTLA4 antagonistic antibody. Similarly, the variable regions of the antibody of the invention can be constructed into Fv molecules and coupled to alternative ligands such as those illustrated in the cited article. Higgins et al., J. Infect Disease 166 (1992), 198-202, described a hetero-conjugated antibody composed of OKT3 cross-linked to an antibody directed to a specific sequence in the V3 region of GP120. Such hetero-conjugate antibodies can also be constructed using at least the variable regions contained in the antibody of the invention methods. Additional examples of specific antibodies include those described by Fanger et al., Cancer Treat. Res. 68 (1993), 181-194 and by Fanger et al., Crit. Rev. Immunol. 12 (1992), 101-124. Conjugates that are immunotoxins including conventional antibodies have been widely described in the art. The toxins may be coupled to the antibodies by conventional coupling techniques or immunotoxins containing protein toxin portions can be produced as fusion proteins. The antibodies of the present invention can be used in a corresponding way to obtain such immunotoxins. Illustrative of such immunotoxins are those described by Byers et al., Seminars Cell. Biol. 2 (1991), 59-70 and by Fanger et al., Immunol. Today 12 (1991), 51-54.

The above described fusion proteins may further comprise a cleavable linker or cleavage site for proteases. These spacer moieties, in turn, can be either insoluble or soluble (Diener et al., Science 231 (1986), 148) and can be selected to enable drug release from the antigen at the target site.

Examples of therapeutic agents, which can be coupled to the antibodies, and antigens of the present invention for immunotherapy are drugs, radioisotopes, lectins, and toxins. The drugs which can be conjugated to the antibodies and antigens of the present invention include compounds, which are classically referred to as drugs such as mitomycin C, daunorubicin, and vinblastine. In using radioisotopically conjugated antibodies or antigens of the invention for, e.g., tumor immunotherapy, certain isotopes may be more preferable than others depending on such factors as leukocyte distribution as well as stability and emission.

Some emitters may be preferable to others. In general, alpha and beta particle emitting radioisotopes are preferred in immunotherapy. Preferred are short range, high energy a emitters such as ²¹²Bi. Examples of radioisotopes, which can be bound to the antibodies, or antigens of the invention for therapeutic purposes are ¹²⁵I, ¹³¹I, ⁹⁰Y, ⁶⁷Cu, ²¹²Bi, ²¹²At, ²¹¹Pb, ⁴⁷Sc, ¹⁰⁹Pd and ¹⁸⁸Re. Other therapeutic agents, which can be coupled to the antibody or antigen of the invention, as well as ex vivo and in vivo therapeutic protocols, are known, or can be easily ascertained, by those of ordinary skill in the art.

Preferably, MAGEA3 binding antibodies or binding fragments thereof as they are mentioned here include the above-mentioned specific MAGEA3 antibodies which have been characterized inter alia by SEQ ID Nos. 1 to 100 and which may then be further modified as described herein to yield fragments, variants etc.

All of these specific individual MAGEA3 binding antibodies or fragments thereof have in common that they have either been directly obtained from patients which suffer from a MAGEA3 expressing tumor and which have been classified as complete or at least partial responders or that they have been derived from antibodies of such patients. They thus are either monoclonal human patient-derived antibodies or monoclonal chimeric, humanized or human antibodies, binding fragments thereof and their variants, which preserve the essential properties of the monoclonal human patient-derived antibodies.

It seems justified to assume that such antibodies and binding fragments thereof will be particularly effective in the treatment of MAGEA3 and/or MAGEA6 expressing tumors or even other cancer types. The effectiveness of such antibodies may result from their capability to induce an immune response against the tumor by e.g. activating CD4⁺, CD8⁺ cytotoxic T cells. Certain antibodies such as 122G3 and 32H2 and binding fragments, variants etc., which are derived thereof, may be preferred as they seem to have a rather high affinity (31 and 19 pM, respectively). Further, they seem to preferentially recognize MAGEA3 over MAGEA1, MAGEA2, MAGEA4 as well as MAGEA10.

The present invention further relates to nucleic acid molecules encoding for such antibodies, to nucleic acid molecules encoding for the variable light and/or heavy chains thereof and to nucleic acid molecules encoding for the CDR1, CDR2 and/or CDR3 of the variable light and/or heavy chains thereof.

The present invention further relates to vectors comprising such nucleic acid molecules and/or such vectors.

The present invention also relates to pharmaceutical compositions comprising any of the aforementioned MAGEA3 binding antibodies or binding fragments thereof.

The present invention further relates to pharmaceutical compositions comprising any of the aforementioned MAGEA3 binding antibodies or binding fragments thereof for use in treating hyper-proliferative diseases, in particular tumors, which express MAGEA3 and/or MAGEA6.

The present invention further relates to the use of any of the aforementioned MAGEA3 binding antibodies or binding fragments thereof in the manufacture of a medicament for treating hyper-proliferative diseases, in particular tumors, which express MAGEA3 and/or MAGEA6.

The present invention further relates to methods of treating hyper-proliferative diseases, in particular tumors which express MAGEA3 and/or MAGEA6 by administering to patients any of the aforementioned MAGEA3 binding antibodies or binding fragments thereof.

As mentioned, the invention also relates in some embodiment to nucleic acid molecules encoding antibodies and binding fragments thereof, vectors comprising such nucleic acid molecules and host cells comprising such nucleic acid sequences and vectors.

The antibodies and binding fragments thereof may be encoded by a single nucleic acid (e.g., a single nucleic acid comprising nucleotide sequences that encode the light and heavy chain polypeptides of the antibody), or by two or more separate nucleic acids, each of which encodes a different part of the antibody or antibody fragment. In this regard, the invention provides one or more nucleic acids that encode any of the foregoing antibodies, or binding fragments (e.g., any of the foregoing light or heavy chain variable regions of SEQ ID NOs: 3, 13, 23, 33, 4, 14, 24, 34 or any of the CDRs of SEQ ID Nos.: 5, 15, 25, 35, 6, 16, 26, 36, 7, 17, 27, 37, 8, 18, 28, 38, 9, 19, 29, 39, 10, 20, 30, 40). The nucleic acid molecules may be DNA, cDNA, RNA and the like.

According to one aspect of the invention, the invention provides a nucleic acid that encodes a heavy chain variable region of an antibody or a portion thereof. Exemplary nucleic acid sequences are provided in SEQ ID Nos: 1, 11, 21, 31. The invention also provides a nucleic acid that encodes a light chain variable region of an antibody or a portion thereof. Exemplary nucleic acid sequences are provided in SEQ ID Nos.: 2, 12, 22, 32.

Also encompassed by the invention are nucleic acids encoding any of the foregoing amino acid sequences of the light or heavy chains that comprise one or more conservative substitutions (e.g., 1, 2, 3, 4, 5, 6, 7, 8, 9, 10, 11, 12, 13, 14, or conservative substitutions), as discussed with respect to the antibody and antibody fragment of the invention, where the antibody or fragment comprising the substitution has the same or substantially the same affinity and specificity of epitope binding as one or more of the exemplary antibodies, fragments and sequences disclosed herein.

Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally associated or heterologous promoter regions.

The nucleic acids described herein can be inserted into vectors, e.g., nucleic acid expression vectors and/or targeting vectors. Such vectors can be used in various ways, e.g., for the expression of an antibody or a binding fragment in a cell or transgenic animal. Accordingly, the invention provides a vector comprising any one or more of the nucleic acids of the invention. A “vector” is any molecule or composition that has the ability to carry a nucleic acid sequence into a suitable host cell where synthesis of the encoded polypeptide can take place. Typically and preferably, a vector is a nucleic acid that has been engineered, using recombinant DNA techniques that are known in the art, to incorporate a desired nucleic acid sequence (e.g., a nucleic acid of the invention). Desirably, the vector is comprised of DNA. However, vectors that are not based on nucleic acids, such as liposomes, are also known in the art and can be used in connection with the invention. The inventive vector can be based on a single type of nucleic acid (e.g., a plasmid) or non-nucleic acid molecule (e.g., a lipid or a polymer). Alternatively, the vector can be a combination of a nucleic acid and a non-nucleic acid (i.e., a “chimeric” vector). For example, a plasmid harboring the nucleic acid can be formulated with a lipid or a polymer as a delivery vehicle. Such a vector is referred to herein as a “plasmid-lipid complex” and a “plasmid-polymer” complex, respectively. The inventive gene transfer vector can be integrated into the host cell genome or can be present in the host cell in the form of an episome.

Vectors are typically selected to be functional in the host cell in which the vector will be used (the vector is compatible with the host cell machinery such that amplification of the gene and/or expression of the gene can occur). A nucleic acid molecule encoding an antibody or binding fragment thereof may be amplified/expressed in prokaryotic, yeast, insect (baculovirus systems) and/or eukaryotic host cells. Selection of the host cell will depend in part on whether the antibody or fragment is to be post-transitionally modified (e.g., glycosylated and/or phosphorylated). If so, yeast, insect, or mammalian host cells are preferable.

Expression vectors typically contain one or more of the following components (if they are not already provided by the nucleic acid molecules): a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

The invention in some aspects further provides a cell (e.g., an isolated or purified cell) comprising a nucleic acid or vector of the invention. The cell can be any type of cell capable of being transformed with the nucleic acid or vector of the invention so as to produce a polypeptide encoded thereby. The cell is preferably the cell of a mammal, such as a human, and is more preferably a hybridoma cell, an embryonic stem cell, or a fertilized egg. The embryonic stem cell or fertilized egg may not be a human embryonic stem cell or a human fertilized egg.

The host cells may be prokaryotic host cells (such as E. coli) or eukaryotic host cells (such as a yeast cell, an insect cell, or a vertebrate cell). The host cell, when cultured under appropriate conditions, expresses an antibody or binding fragment which can subsequently be collected from the culture medium (if the host cell secretes it into the medium) or directly from the host cell producing it (if it is not secreted). Selection of an appropriate host cell will depend upon various factors, such as desired expression levels, polypeptide modifications that are desirable or necessary for activity, such as glycosylation or phosphorylation, and ease of folding into a biologically active molecule. A number of suitable host cells are known in the art and many are available from the American Type Culture Collection (ATCC), Manassas, Va. Examples include mammalian cells, such as Chinese hamster ovary cells (CHO) (ATCC No. CCL61) CHO DHFR-cells (Urlaub et al. Proc. Natl. Acad. Sci. USA 97, 4216-4220 (1980)), human embryonic kidney (HEK) 293 or 293T cells (ATCC No. CRL1573), 3T3 cells (ATCC No. CCL92), or PER.C6 cells.

The cell comprising the nucleic acid or vector of the invention can be used to produce the antibody or binding fragment thereof, or a portion thereof (e.g., a heavy chain sequence, or a light chain sequence encoded by the nucleic acid or vector). After introducing the nucleic acid or vector of the invention into the cell, the cell is cultured under conditions suitable for expression of the encoded sequence. The antibody, antigen binding fragment, or portion of the antibody then can be isolated from the cell.

The TAA binding antibodies or binding fragments thereof as well as the compounds capable of activating the immune system can be formulated in compositions, especially pharmaceutical compositions. Such compositions comprise a therapeutically or prophylactically effective amount of an antibody or binding fragment thereof and/or of compounds capable of activating the immune system in admixture with a suitable carrier, e.g., a pharmaceutically acceptable agent.

Pharmaceutically acceptable agents for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in liquid form or in a lyophilized or freeze-dried form and may include one or more lyoprotectants, excipients, surfactants, high molecular weight structural additives and/or bulking agents (see for example U.S. Pat. Nos. 6,685,940, 6,566,329, and 6,372,716).

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980, which is incorporated herein by reference.

Pharmaceutical compositions described herein can be formulated for controlled or sustained delivery in a manner that provides local concentration of the product (e.g., bolus, depot effect) and/or increased stability or half-life in a particular local environment. The compositions can include the formulation of antibodies, binding fragments, nucleic acids, or vectors of the invention with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection.

Both biodegradable and non-biodegradable polymeric matrices can be used to deliver compositions of the present invention, and such polymeric matrices may comprise natural or synthetic polymers. Biodegradable matrices are preferred. The period of time over which release occurs is based on selection of the polymer. Typically, release over a period ranging from between a few hours and three to twelve months is most desirable.

Alternatively or additionally, the compositions can be administered locally via implantation into the affected area of a membrane, sponge, or other appropriate material on to which an antibody, binding fragment, nucleic acid, or vector of the invention has been absorbed or encapsulated. Where an implantation device is used, the device can be implanted into any suitable tissue or organ, and delivery of an antibody, binding fragment, nucleic acid, or vector of the invention can be directly through the device via bolus, or via continuous administration, or via catheter using continuous infusion.

A pharmaceutical composition comprising a binding antibody or binding fragment thereof and/or compounds capable of activating the immune system can be formulated for inhalation, such as for example, as a dry powder. Inhalation solutions also can be formulated in a liquefied propellant for aerosol delivery. In yet another formulation, solutions may be nebulized.

Certain formulations containing antibodies or binding fragments thereof and/or compounds capable of activating the immune system can be administered orally. Formulations administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of a selective binding agent. Diluents, flavorings, low melting point waxes, vegetable oils, lubricants, suspending agents, tablet disintegrating agents, and binders also can be employed.

In some embodiments, the pharmaceutical compositions as mentioned before may comprise a MAGEA3 binding antibody or binding fragments thereof but may not comprise a compound capable of stimulating the immune system. The term “compound capable of stimulating the immune system” is to be understood as described hereinafter and particularly includes CD40L or anti-CD40 agonistic antibodies such as CP-870,893 or SGN-40 and (iii) anti-CTLA4 antagonistic antibodies such as Tremelimumab and Ipilimumab.

In some other embodiments, the pharmaceutical compositions as mentioned before may comprise a MAGEA3 binding antibody or binding fragments thereof as the sole pharmaceutically active agent.

Further the MAGEA3 binding antibodies or binding fragments thereof and all types of pharmaceutical compositions as contemplated herein can be administered in methods of treating patients suffering from hyper-proliferative diseases and/or preventing individuals from developing hyper-proliferative diseases. The term “hyper-proliferative disease” refers to diseases, which are commonly designated as cancer or tumors.

Unless stated otherwise, the terms “cancer” and “tumor” are used interchangeably herein. These terms in particular relate but are not limited to cancer and tumors selected from the group comprising basal cell carcinoma; bladder cancer; bone cancer such as osteosarcoma; central nervous system tumors such as cerebellar astrocytoma, cerebral astrocytoma/malignant glioma, craniopharyngioma, ependymoblastoma, ependymoma, medulloblastoma, medulloepithelioma, pineal parenchymal tumors of intermediate differentiation, primitive neuroectodermal tumors, pineoblastoma and spinal cord tumors; Burkitt's lymphoma; breast cancer; cervical cancer; chronic myelogenous leukemia; colon cancer; rectal cancer; colorectal cancer; esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; T-cell lymphoma; mesothelioma; multiple myeloma/plasma cell neoplasm; myeloid leukemia; multiple myeloma; nasopharyngeal cancer; neuroblastoma; small cell lung cancer; non-small cell lung cancer; oropharyngeal cancer; osteosarcoma; ovarian cancer; pancreatic cancer; parathyroid cancer; penile cancer; pharyngeal cancer; phaeochromocytoma; pituitary tumor; prostate cancer; renal cell cancer; respiratory tract carcinoma; retinoblastoma; skin cancer (melanoma); small intestine cancer; soft tissue sarcoma; squamous cell carcinoma; squamous neck cancer; stomach (gastric) cancer; testicular cancer; throat cancer; thyroid cancer; transitional cell cancer of the renal pelvis and ureter; urethral cancer; uterine cancer; vaginal cancer; vulvar cancer and Wilms tumor.

Treatment of melanoma, breast cancer, ovarian cancer, non-small cell lung cancer, multiple myeloma and/or pancreatic cancer may be particularly effective when using the MAGEA3 binding antibodies or binding fragments thereof or pharmaceutical compositions as described herein.

As mentioned before, in other embodiments of the first aspect of the invention, the MAGEA3 binding antibodies or binding fragments thereof as described herein are used as a diagnostic tool, e.g. for diagnosing patients suffering from hyperproliferative diseases as mentioned herein. It can be preferred to use such antibodies to diagnose the occurrence and/or development of e.g. hyperproliferative diseases, which express MAGEA3 and/or MAGEA6. Such hyperproliferative diseases may include melanoma, breast cancer, ovarian cancer, non-small cell lung cancer, multiple myeloma and/or pancreatic cancer. The MAGEA3 binding antibodies and binding fragments thereof may preferably be used to identify hyperproliferative diseases such as the aforementioned cancers which are primarily characterized by MAGEA3 and/or MAGEA6 and optionally MAGEA2 overexpression over cancers which are characterized in addition or solely by MAGEA4, MAGEA1 or MAGEA10 overexpression. If a treatment is available that primarily is effective for hypreproliferative disease such as cancers being characterized by MAGEA3 and/or MAGEA6 and optionally MAGEA2 overexpression, the antibodies of the present invention and antibodies and binding fragments derived therefrom can be used for stratification of patient populations in clinical trials or as companion diagnostic, i.e. selecting patients for which the treatment will be effective,

In one embodiment, the present invention thus relates to a diagnostic composition comprising the MAGEA3 binding antibodies or binding fragments described herein. Such diagnostic compositions may be for use in diagnosing occurrence and/or development of e.g. hyperproliferative diseases, which express MAGEA3 and/or MAGEA6 and optionally MAGEA2. Such hyperproliferative diseases may include melanoma, breast cancer, ovarian cancer, non-small cell lung cancer, multiple myeloma and/or pancreatic cancer.

In another embodiment, the invention relates to MAGEA3 binding antibodies or binding fragments thereof as described herein for use in diagnosing hyperproliferative diseases. These diseases may express MAGEA3 and/or MAGEA6 and optionally MAGEA2. Such hyperproliferative diseases may include melanoma, breast cancer, ovarian cancer, non-small cell lung cancer, multiple myeloma and/or pancreatic cancer.

In another embodiment, the present invention relates to the use of MAGEA3 binding antibodies or binding fragments thereof as described herein in the manufacture of a composition and/or medicament for diagnosing hyperproliferative diseases. These diseases may express MAGEA3 and/or MAGEA6 and optionally MAGEA2. Such hyperproliferative diseases may include melanoma, breast cancer, ovarian cancer, non-small cell lung cancer, multiple myeloma and/or pancreatic cancer.

In yet another embodiment, the present invention relates to a method of diagnosing a hyperproliferative disease in a human or animal being by using MAGEA3 binding antibodies or binding fragments thereof as described herein. These diseases may express MAGEA3 and/or MAGEA6 and optionally MAGEA2. Such hyperproliferative diseases may include melanoma, breast cancer, ovarian cancer, non-small cell lung cancer, multiple myeloma and/or pancreatic cancer.

Such methods and uses may be conducted outside the human body. To this extent a sample, e.g. a tissue sample may be obtained from an individual, which is suspected to suffer from e.g. imminent or ongoing cancer development. This sample will then be tested e.g. for MAGEA3 and/or MAGEA6 expression. Typically, this determination will include comparing the expression level of e.g. MAGEA3 and/or MAGEA6 with the respective expression levels of samples, which have been obtained from either healthy individuals or healthy tissue of the same individual.

The invention thus relates e.g. to a method of diagnosing a hyperproliferative disease in a human or animal individual comprising at least the steps of:

a) Testing a sample of said human or animal individual for expression of at least MAGEA3 using a MAGEA3 binding antibody or binding fragment as described herein;

b) Comparing with expression of at least MAGEA3 in a control sample;

c) ‘Determining the occurrence and/or likely development of a hyperproliferative disease by comparing the data obtained in steps a) and b).

Steps a) and b) may preferably be conducted outside the human or animal body. A sample may be tissue, organs, etc. A control sample may be as described before.

Such methods and uses may, however, also be conducted directly on the human body. To this extent the antibodies may be labeled with a detectable marker, be administered to an individual, which is suspected to suffer from e.g. imminent or ongoing cancer development and localization of the antibody as well as expression levels of MAGEA3 and/or MAGEA6 will be directly determined in the individual.

The invention thus relates e.g. to a method of stratifying a patient population for e.g. clinical trials for testing treatment of a hyperproliferative disease in a human or animal individual comprising at least the steps of:

a) Testing a sample of said human or animal individual for expression of at least MAGEA3 using a MAGEA3 binding antibody or binding fragment as described herein;

b) Comparing with expression of at least MAGEA3 in a control sample;

c) Selecting the patient population by comparing the data obtained in steps a) and b).

Steps a) and b) may preferably be conducted outside the human or animal body. A sample may be tissue, organs, etc. A control sample may be as described before.

The invention also relates to a method of data acquisition comprising at least the steps of:

a) Administering a MAGEA3 binding antibody or binding fragment thereof as described herein to a human or animal individual;

b) Determining the distribution of said MAGEA3 binding antibody or binding fragment thereof in said human or animal individual; and

c) Determining expression of at least MAGEA3 in said human or animal individual.

As mentioned above, in a second aspect the present invention relates to a combination of TAA binding antibodies such as the MAGEA3-binding antibodies or binding fragments thereof as described herein and a compound capable of stimulating the immune system.

This second aspect of the present invention is inter alia based on the experimental finding that mice with a syngeneic NY-ESO-1 positive colon tumor which were treated with 5-FU display infiltration of CD4⁺, CD8⁺ T-cells after administration of NY-ESO-1 binding antibody 12D7 (having a variable light chain of SEQ ID No. 109 ad and a variable heavy chain of SEQ ID No. 110) and that this effect is more pronounced upon additional administration of anti-CD40 agonistic antibodies. As a consequence of these treatments, tumor size is reduced. This NY-ESO-1 binding antibody as well as other specific NY-ESO-1 binding antibodies and fragments thereof are described in detail in EP 11 150 527.7.

Without wanting to be held to this hypothesis, it is assumed that administration of TAA binding antibodies such as the CT-antigen NY-Eso-1 binding antibody 12D7 triggers an immune response, which from a therapeutic perspective (e.g. in terms of tumor destruction) is localized at the site of tumor. It seems that this type of localized immune response can be further augmented and/or prolonged by administration of compounds, which are capable of activating the immune system such as the CD40 agonistic antibodies. It is assumed that the same type of cooperative action can be observed if e.g. MAGEA3 binding antibodies or binding fragments thereof as they are disclosed herein are combined with such compounds, which are capable of activating the immune system.

This combined approach of using very selective tumor targeting agents (i.e. the TAA binding antibodies such as the MAGEA3 binding antibodies or binding fragments thereof as described herein) with what may be designated as broad band immuno-modulating agents (i.e. compounds capable of stimulating an immune response) and even non-specific cytotoxic agents may provide several advantages that may significantly improve disease therapy.

Standard chemotherapy with compounds such as 5-FU, therapies focusing on general immuno-modulators e.g. via toll-7 or toll-9 receptor agonists, CD-40 receptor agonists, anti-CTLA-4 antagonistic antibodies and even more targeted therapies involving therapeutic antibodies directed against the EGF-Receptor or HER-2 receptor suffer from various side effects.

Chemotherapy with cytotoxic agents affects dividing cells in general. Immunomodulators enhance other non-tumor directed immune reactions as well as adverse autoimmune reactions. Antibody addressed EGF-Receptors or HER-2 receptors are of functional relevance not only in tumor tissue but in other differentiated normal cells as well e.g. of the heart.

These properties lead to “off-target” (the target being the tumor) side effects, which can e.g. limit the dosage and thus the effectiveness of these otherwise therapeutically extremely important therapeutic principles.

By the use of TAA binding antibodies and preferably of CT antigen binding antibodies such as the MAGEA3 binding antibodies or binding fragments thereof as described herein which may be monoclonal human patient-derived antibodies as described hereinafter, the therapeutically important effects of systemically active immune-modulators may be boosted as these activities seem to be more limited to the therapeutic areas of interest, namely the tumor tissue which is pre-selected through the TAA binding antibodies such as MAGEA3 binding antibodies. This assumed pre-selection of the therapeutic area of interest, namely the tumor tissue, by TAA binding antibodies such as the MAGEA3 binding antibodies or binding fragments thereof as described herein and the focusing of the broad band activity of immune-modulating agents to these areas of therapeutic interest should limit off-target related adverse events at least to some extent. This should in turn allow e.g. using immuno-modulating agents such as anti-CD40 agonistic antibodies in higher concentrations than usual and to thus benefit to a greater extent from their therapeutic potential. One may also envisage more effective dosage regimens such as shortened intervals for subsequent administration of the pharmaceutically active agents.

Given that the initial immune response is selectively targeted by the TAA binding antibody such as the MAGEA3 binding antibodies or binding fragments thereof as described herein to tumor tissue only, the additional augmentation seems to also preferentially only effect the tumor tissue only. Such a localized integrated tumor specific immune response may be particularly effective if chemotherapy with e.g. 5-FU makes the TAAs such as the MAGEA3 binding antibodies or binding fragments thereof as described herein readily accessible for the TAA binding antibody.

Based on the above observations, it seems justified that the effects observed for the combination of 5-FU, the NY-ESO-1 binding antibody 12D7 and anti-CD40 agonistic antibodies may also apply for other CT-antigen binding antibodies or TAA binding antibodies in general such as the MAGEA3 binding antibodies or binding fragments thereof as described herein, for other activators of the immune system such as CD40L, anti-OX40 agonistic antibodies, anti-CD137 agonistic antibodies, anti-CTLA4 antagonistic antibodies, anti PD-1 antagonistic antibodies or anti-CD25 antagonistic antibodies and for other cellular stress inducing therapies such as radiation.

The invention in one embodiment of the second aspect is therefore directed to a pharmaceutical composition comprising at least one tumor associated antigen (TAA) binding antibody or binding fragment thereof such as the MAGEA3 binding antibodies or binding fragments thereof as described herein and at least one compound capable of activating the immune system. The combination of such pharmaceutically active agents may also be comprised within a kit of pharmaceutical compositions as it is described hereinafter.

It should be understood that the term “kit” indicates that the invention considers the treatment of e.g. hyper-proliferative diseases as mentioned hereinafter by combinations of pharmaceutically active agents and that these pharmaceutically active agents (e.g. an MAGEA3 binding antibody, an anti-CD40 agonistic antibody and/or an anti-CTLA4 antagonistic antibody) do not need to be combined within a single pharmaceutical dosage form. In fact, it may be advantageous to actually use e.g. an MAGEA3 binding antibody and an anti-CD40 agonistic antibody in the form of separately provided pharmaceutical dosage forms as this will allow accounting e.g. for different pharmacokinetic properties of these antibodies during treatment. The term “kit” therefore is also not to be understood as referring to e.g. necessarily simultaneously offering separate pharmaceutical dosage forms, which comprise the pharmaceutically active agent even though such type of offering is not excluded. The term “kit” indicates that the invention focuses on a use of a combination of different pharmaceutically active agents during therapy and that this combination may e.g. be offered as separate single pharmaceutical dosage forms which can then be used in e.g. a method or use in accordance with the invention.

The present invention in one embodiment of the second aspect thus also relates to a combination of at least one tumor associated antigen (TAA) binding antibody or binding fragment thereof such as the MAGEA3 binding antibodies or binding fragments thereof as described herein and at least one compound capable of activating the immune system for use in treating a disease such as a hyper-proliferative disease. The TAA binding antibody or binding fragments thereof and the at least one compound capable of activating the immune system may be selected as described hereinafter. The components of such combination may be used simultaneously or sequentially for treatment of e.g. hyper-proliferative diseases.

The term “Tumor Associated Antigen (TAA)” is used as described above. Examples of TAAs in general can be taken from Table of EP 11 150 527.7.

The term “compound capable of activating the immune system” refers to a pharmaceutically acceptable compound which is capable of prolonging and/or augmenting an initial immune response which has been triggered by a TAA binding antibody or binding fragment thereof such as the MAGEA3 binding antibodies or binding fragments thereof as described herein.

Such compounds can include compounds which are known to stimulate or at least co-stimulate a humoral or cellular immune response even if no TAA binding antibody or binding fragment thereof such as the MAGEA3 binding antibodies or binding fragments thereof as described herein has been administered prior to, simultaneous with or after administration of such compounds.

Preferably, the term “compound capable of activating the immune system” thus refers to a pharmaceutically acceptable compound which stimulates or at least co-stimulates e.g. maturation of Antigen Presenting Cells (APC) including e.g. dendritic cells, macrophages, neutrophils and eosinophils, T-cell activation, T-cell proliferation including e.g. CD4⁺ helper T-cell and/or CD8⁺ cytotoxic T-cell proliferation, expansion of T-cells, maintenance of memory T-cells and/or proliferation of NK cells. It is to be understood that for the purposes of the present Invention TAA binding antibodies or binding fragments thereof such as CT-antigen binding antibodies or binding fragments thereof and in particular the MAGEA3 binding antibodies or binding fragments thereof as described herein are not considered as representatives of “compounds capable of activating the immune system.”

The aforementioned “compounds capable of activating the immune system” may exert their activating function on the immune system through different mechanisms.

For example, “compounds capable of activating the immune system” may comprise natural components of the immune system which are known to be involved in the stimulation or at least co-stimulation of the aforementioned activities such as e.g. maturation of Antigen Presenting Cells (APC) including e.g. dendritic cells, macrophages, neutrophils or eosinophils, T-cell activation, T-cell proliferation including e.g. CD4⁺ helper T-cell and/or CD8⁺ cytotoxic T-cell proliferation, expansion of T-cells, maintenance of memory T-cells and/or proliferation of NK cells. Such natural components of the immune system, which according to the invention are “compounds capable of activating the immune system” include CD40, CD40 Ligand (CD40L), CD80, CD80 Ligand, C86 and CD86 Ligand, DRS, B7, OX40, CD137, cytokines such as IL-2, IL-6, IL-8, IL-10, IL-12, TNF-a, MIP-1a, and others. These components form a subgroup of “compounds capable of activating the immune system” and may be designated as “natural stimulants or at least co-stimulants of the immune system”. A preferred representative of this subgroup is CD40L.

“Compounds capable of activating the immune system” may, however, also comprise compounds which do not constitute natural components of the immune system but which induce and/or increase the activity of the aforementioned natural components of the immune system, i.e. have an agonistic effect on “natural stimulants or at least co-stimulants of the immune system”. This subgroup of “compounds capable of activating the immune system” may be designated as “agonistic activators of natural stimulants or at least co-stimulants of the immune system”. Preferred embodiments of this latter subgroup comprise anti-CD40 agonistic antibodies such as CP-870,893, SGN-40, FGK45.5 or a humanized form thereof, anti-OX40 agonistic antibodies such as OX86, anti-CD137 agonistic antibodies such as BMS-663513 and others. Information on such factors and antibodies can be taken inter alia from Weiner et al., (2010), Nature Reviews, 10, 317-327, Fonsatti et al., (2010), Seminars in Oncology, 37(5), 517-523 or Vonderheide (2007), Molecular Pathways, 13(4), 1083-1088.

Other “compounds capable of activating the immune system” include compounds which release an inhibitory effect of natural components of the immune system on the aforementioned activities such as e.g. maturation of Antigen Presenting Cells (APC) including e.g. of dendritic cells, macrophages, neutrophils or eosinophils, T-cell activation, T-cell proliferation including e.g. CD4⁺ helper T-cell and/or CD8⁺ cytotoxic T-cell proliferation, expansion of T-cells, maintenance of memory T-cells and/or proliferation of NK cells. Examples of such natural components of the immune system, which have an inhibitory or at least co-inhibitory effect on the aforementioned activities, include e.g. CTLA4, CD25 PD-1 or sMICA. This further subgroup of “compounds capable of activating the immune system” may be designated as “antagonistic effectors of natural inhibitors or at least co-inhibitors of the immune system”. Examples of “antagonistic effectors of natural inhibitors or at least co-inhibitors of the immune system” include anti-CTLA4 antagonistic antibodies such as Tremelimumab and Ipilimumab, anti-CD25 antagonistic antibodies such as Daclizumab and anti-PD1 antagonistic antibodies such as CT-011. Information on such factors and antibodies can be taken inter alia from Weber, (2008), The Oncologist, 13(suppl 4), 16-25 or Fonsatti et al., (2010), Seminars in Oncology, 37(5), 517-523.

In a preferred embodiment of the invention “compounds capable of activating the immune system” are selected from CD40L, anti-CD40 agonistic antibodies including CP-870,893, and SGN-40 and anti-CTLA4 antagonistic antibodies including Tremelimumab and Ipilimumab.

It is to be understood that if antibodies such as anti-CD40 agonistic antibodies including CP-870,893, and SGN-40 or anti-CTLA4 antagonistic antibodies including Tremelimumab and Ipilimumab are used as compounds capable of activating the immune system, they may be used as binding fragments, variants etc. of the respective antibody.

Other “compounds capable of activating the immune system” include compounds, which are known to act on the innate immune system such as activators of Toll-like receptors including Toll-like receptors, 2, 3, 4, 5, 7, 8, and 9. Such compounds include bacterial lipoprotein, LPS, double-stranded RNA, poly I:C (polyinosinic polycytidylic acid), bacterial flagellin resiquimod (R848) and CpG-ODN.

As mentioned above, the TAA binding antibodies or binding fragments thereof such as the MAGEA3 binding antibodies or binding fragments thereof as described herein may be combined with compounds capable of activating the immune system in different fashions.

Thus, a TAA binding antibody such as the MAGEA3 binding antibodies described herein may be combined with natural stimulants or at least co-stimulants of the immune system, agonistic activators of natural stimulants or at least co-stimulants of the immune system or with antagonistic effectors of natural inhibitors or at least co-inhibitors of the immune system.

A specific example would be the combination of a MAGEA3 binding antibody as disclosed herein (such as 122G3 or 32H2) with anti-CD40 agonistic antibodies such as CP-870,893 or SGN-40, anti-OX40 agonistic antibodies such as OX86 and/or anti-CD137 agonistic antibodies such as BMS-663513.

Another specific example would be the combination of a MAGEA3 binding antibody as disclosed herein (such as 122G3 or 32H2) with anti-CTLA4 antagonistic antibodies such as Tremelimumab or Ipilimumab and/or anti-CD25 antagonistic antibodies such as Daclizumab.

However, a TAA binding antibody such as the MAGEA3 binding antibodies or binding fragments thereof as described herein may also be combined with e.g. (i) natural stimulants or at least co-stimulants of the immune system or agonistic activators of natural stimulants or at least co-stimulants of the immune system and (ii) with antagonistic effectors of natural inhibitors or at least co-inhibitors of the immune system.

A specific example would be the combination of a MAGEA3 binding antibody or binding fragment thereof as disclosed herein (such as 122G3 or 32H2) with anti-CD40 agonistic antibodies such as CP-870,893 or SGN-40 and with anti-CTLA4 antagonistic antibodies such as Tremelimumab or Ipilimumab.

Other examples may further include OX86, BMS-663513, CT-011 and/or Daclizumab.

Further compounds, which are known to act on the innate immune system such as activators of Toll-like receptors 1, 2, 3, 4, 5, 7, 8, and 9 may be included.

A preferred embodiment comprises a combination of a MAGEA3 binding antibody as disclosed herein (such as 122G3 or 32H2) with anti-CD40 agonistic antibodies such as CP-870,893 or SGN-40 as the sole pharmaceutically active agents.

Another preferred embodiment comprises a combination of a MAGEA3 binding antibody as disclosed herein (such as 122G3 or 30H10) with anti-CTLA4 antagonistic antibodies such as Tremelimumab or Ipilimumab as the sole pharmaceutically active agents.

Yet another preferred embodiment comprises a combination of a MAGEA3 binding antibody as disclosed herein (such as 122G3 or 32H2) with anti-CD40 agonistic antibodies such as CP-870,893 or SGN-40 and with anti-CTLA4 antagonistic antibodies such as Tremelimumab or Ipilimumab as the sole pharmaceutically active agents.

It has been mentioned above that the different pharmaceutically active principles such as the MAGEA3 binding antibody or binding fragment thereof, natural stimulants or at least co-stimulants of the immune system, agonistic activators of natural stimulants or at least co-stimulants of the immune system or antagonistic effectors of natural inhibitors or at least co-inhibitors of the immune system may be combined within multi-specific antibodies such as bi-specific antibodies or binding fragments thereof. This will be illustrated for the specific example of a MAGEA3 binding antibody and an anti-CD40 agonistic or an anti-CTLA4 antagonistic antibody or binding fragment thereof. However, it will be understood that this principle can be extended to other compounds capable of activating the immune system as well.

Thus, a portion of a MAGEA3 binding antibody or binding fragment thereof and (i) a portion of an anti-CD40 agonistic antibody or binding fragment thereof or (ii) a portion of an anti-CTLA4 antagonistic antibody or binding fragment thereof may be combined in a bi-specific antibody.

Such bispecific antibodies or fragments can be of several configurations. For example, bispecific antibodies may resemble single antibodies (or antibody fragments) but have two different antigen binding sites (variable regions). Bispecific antibodies can be produced by chemical techniques (Kranz et al. (1981), Proc. Natl. Acad. Sci. USA, 78: 5807) or by recombinant DNA techniques. Bispecific antibodies can have binding specificities for at least two different epitopes, at least one of which is an epitope of the tumor-associated antigen for which the antibody has been identified. The antibodies and binding fragments can also be heteroantibodies. Heteroantibodies are two or more antibodies, or antibody binding fragments (Fab) linked together, each antibody or fragment having a different specificity.

The use of such bispecific antibodies can have the advantage that the augmentation and/or prolongation of the initial localized immune response which is assumed to be triggered by the TAA binding antibody is confined to the tumor as precisely as possible.

This concept can, of course, be extended to tri-specific antibodies which would comprise e.g. a portion of a MAGEA3 binding antibody or binding fragment thereof, a portion of an anti-CD40 agonistic antibody or binding fragment thereof and a portion of an anti-CTLA4 antagonistic antibody or binding fragment thereof.

As has been set out before, the aforementioned combinations may be provided in the form of a single pharmaceutical composition which would be the case e.g. for a bispecific antibody or they may be provided as a kit of pharmaceutical compositions.

Where a kit is contemplated, it may comprise the pharmaceutically active agents in separate pharmaceutical compositions in different combinations. This will again be illustrated for the specific example of a cytotoxic agent, a MAGEA3 binding antibody, an anti-CD40 agonistic antibody and an anti-CTLA4 antagonistic antibody. However, it will be understood that this principle can be adapted accordingly to other combinations.

In the aforementioned example, the kit may consist of two pharmaceutical compositions, the first pharmaceutical composition comprising the cytotoxic agent and the second pharmaceutical composition comprising a MAGEA3 binding antibody and an anti-CD40 agonistic antibody. This kit would allow to first treating a patient with chemotherapy which is assumed to make (in this case) the MAGEA3 and/or MAGEA6 antigen more readily accessible to the MAGEA3 binding antibody. However, the subsequent administration of the second pharmaceutical composition then ensures simultaneous delivery of both the MAGEA3 binding antibody and the anti-CD40 agonistic antibody. This will allow the anti-CD40 agonistic antibody to display its activity as soon as the MAGEA3 binding antibody has triggered a localized immune response.

In another example, the kit may consist of three pharmaceutical compositions, the first pharmaceutical composition comprising the cytotoxic agent, the second pharmaceutical composition comprising a MAGEA3 binding antibody and the third pharmaceutical composition comprising an anti-CTLA4 antagonistic antibody. This kit would allow to first treating a patient with chemotherapy which is assumed to make (in this case) the MAGEA3 antigen more readily accessible to the MAGEA3 binding antibody. The second and third pharmaceutical compositions could then be administered separately from each other to first trigger a localized immune response by the TAA binding antibody and to allow sufficient time for development of such an immune response before the anti-CTLA4 antagonistic antibody can fully exert its function. However, the anti-CTLA4 antibodies may also help to de-repress already existing MAGEA3 specific T-cells. These cells could be further activated by the subsequent administration of MAGEA3 specific antibodies, which would further strengthen the MAGEA3 binding antibody mediated antigen presentation. For such case the third pharmaceutical composition may be administered before or at least concomitantly with the second pharmaceutical composition.

Such kits could thus be used to e.g. account for the different pharmacokinetic properties of the e.g. respective antibodies by a fine-tuned timely administration.

It has been mentioned above that the efficacy of the aforementioned combinations may be enhanced if the patients receiving such combinations are subjected to a cytotoxic treatment.

The term “cytotoxic treatment” includes chemotherapy, radiation therapy, surgery, hyperthermia and the like. Chemotherapy may include administration of cytotoxic agents such as taxanes including docetaxel and paclitaxel, anthracyclines, cisplatin, carboplatin, 5-fluoro-uracil, gemcitabine, capecitabin, navelbine or zoledronate.

Where chemotherapy and particularly the aforementioned cytotoxic agents are used as cytotoxic treatment, these agents may be included in the pharmaceutical compositions and kits as contemplated above. Preferably, 5-FU may be included.

The combinations of pharmaceutically active agents, which may take the form of pharmaceutical compositions or kits as contemplated herein can be used as medicaments for use in treating patients suffering from hyper-proliferative diseases.

The combinations of pharmaceutically active agents, which may take the form of pharmaceutical compositions or kits as contemplated herein can be also used in the manufacture of medicaments for treating patients suffering from hyper-proliferative diseases.

Further the combinations of pharmaceutically active agents, which may take the form of pharmaceutical compositions or kits as contemplated herein can be administered in methods of treating patients suffering from hyper-proliferative diseases.

The term “hyper-proliferative disease” is used as mentioned above. The use of MAGEA3 binding antibodies or binding fragments thereof as described herein together with compounds capable of stimulating the immune system may be particularly useful for treatment of melanoma, breast cancer, ovarian cancer, non-small cell lung cancer, multiple myeloma and/or pancreatic cancer may be particularly effective.

The efficacy and/or selectivity of pharmaceutical compositions or kits in accordance with the invention towards certain cancers may be increased if different TAA binding antibodies or binding fragments which bind to e.g. different CT antigens are present are combined. Thus, the above-mentioned pharmaceutical compositions or kits may comprise e.g. combinations of MAGEA3 and NY-ESO-1 binding antibodies or binding fragments thereof with e.g. anti CD40 agonistic or binding fragments thereof and/or anti-CTLA4 antagonistic antibodies or binding fragments thereof. Other combinations can be taken from EP 11 150 527.7.

The combination of MAGEA3 binding antibodies or binding fragments thereof with compounds capable of stimulating the immune system can be applied in the form of pharmaceutical compositions as described with respect to pharmaceutically acceptable excipients, routes of administration etc.

If the MAGEA3 binding antibodies or binding fragments thereof are used in combination with compounds capable of stimulating the immune system, such antibodies may take the above-described forms (single chain antibodies etc.) or may be modified with labels as described above.

The combination of MAGEA3 binding antibodies or binding fragments thereof with compounds capable of stimulating the immune system may also be used for methods of treatment as described above and pharmaceutical compositions or kits comprising a combination of MAGEA3 binding antibodies or binding fragments thereof with compounds capable of stimulating the immune system may be applied for the uses described above.

The invention is now described with respect to some examples which are, however, not to be construed as limiting.

### Experiment 1

### Isolation of MAGEA3 Antibodies

### Patient Material

Serum and peripheral blood lymphocytes for the isolation of memory B cells was collected from patients in accordance with the informed consent that was approved by the local Ethical committee(s) and signed by the patient.

Patients suffering from melanoma or breast cancer were vaccinated with full-length human MAGEA3 complexed with an adjuvant. The patients were serum-positive for MAGEA3. Antibodies 32H2 and 34G9 as described herein were derived from the melanoma patient which had received the adjuvant CpG in addition. Antibodies 122G3 and 102G10 as described herein were derived from the patient with breast cancer. Both patients had antibody titers against MAGEA3 in their serum.

### Memory B Cell Culture

Memory B cells were isolated from human peripheral blood monocytic cells with a two-step selection procedure using MACS beads against the pan-B cell marker CD22 (Miltenyi, Bergisch Gladbach, Germany) followed by staining with phycoerythrin-conjugated mAbs anti-human IgD and APC-conjugated antibodies anti human IgM, CD3, CD8, CD56 (Becton Dickinson, Basel, Switzerland). Alternatively, a one-step protocol was applied using phycoerythrin-conjugated mAb anti-human IgD, APC-conjugated mAbs anti-human IgM, CD3, CD56, CD8 and FITC-conjugated mAb anti human CD22 (Becton Dickinson, Basel, Switzerland). Cell sorting was carried out using a MoFlo XDP cell sorter (Beckman Coulter). CD22-positive- and IgM-, IgD-negative B cells were then incubated with EBV containing supernatant obtained from B95-8 cells (in B cell medium containing RPMI 1640 supplemented with 10% fetal calf serum). Cells were seeded in at 10 cells per well in IMDM medium supplemented with CpG 2006 on 30.000 irradiated feeder PBL prepared from voluntary donors.

After 11-14 days of culture the conditioned medium of memory B cell culture was screened for the presence of MAGEA3-specific antibodies by ELISA.

### MAGEA3 ELISA

His-tagged MAGEA3 expressed in bacteria was column purified and used to coat 96 well microplates (Costar, USA). Plates were washed with PBS-T and blocked 1 h at room temperature with PBS containing 2% BSA (Sigma, Buchs, Switzerland). Patient sera, B cell conditioned medium, or recombinant antibody preparations were incubated for 2 h at room temperature. Binding of human IgG to MAGEA3 was determined using a horseradish peroxidase conjugated goat anti human Fc-gamma-specific antibody (Jackson ImmunoResearch, Europe Ltd., Cambridgeshire, UK) followed by measurement of the HRP activity using a TMB substrate solution (TMB, Sigma, Buchs, Switzerland).

### Single Cell-RT-PCR

Single cells obtained from MAGEA3-reactive memory B cell cultures were deposited into a 96 well PCR plate, containing first strand buffer (Invitrogen, LuBioScience, Switzerland). cDNA was prepared using Random hexamer primer (Invitrogen, LuBioScience, Switzerland). PCR amplification of immunoglobulin heavy and light chain variable regions was performed according to standard protocols (Wardemann et al. Science 301, 2003, 1374-1377). Immunoglobulin heavy and light chain variable regions were amplified using a nested PCR approach. 1st round PCR was performed with primers specific for the IgG constant region and primer mixes specific for all signal peptides of heavy and light chain Ig variable region families (Wardemann et al. Science 301, 2003, 1374-1377). Subsequently, nested PCR was performed using primer mixes specific for the immunoglobulin J-regions and the 5′ region of framework 1 of heavy and light chain Ig variable region families. Sequence analysis was carried out to identify the individual antibody clones present in the selected B cell culture. Subsequently, the Ig-variable heavy- and light regions of each antibody clone were cloned into expression vectors providing the constant regions of human IgG1, human Ig-Kappa or human Ig-Lambda. Upon co-transfection of the Ig-heavy- and light expression vectors into HEK 293 cells the antibody clones were produced. Identification of the antibody clone presumably responsible for the MAGEA3-reactivity of the parental B cell culture was performed upon re-screening of the recombinant antibody clones in MAGEA3- and control ELISA. This approach led to identification of 122G3, 32H2, 34G9, and 102G10.

In order to identify and to correct primer encoded sequence mismatches in the Ig-variable region a further PCR amplification using a semi-nested protocol was performed with 2 primer pairs specific for a conserved region of the Ig-heavy- and light chain constant regions as 3′-primers and primer mixes specific for the Ig-signal peptides as 5′-primers. PCR products were cloned into TOPO™ vector (Invitrogen, LuBioScience, Lucerne, Switzerland). Sequence determination of the complete Ig-variable region was carried out and the information was used to design specific primers for the cloning of the authentic human antibody sequence into antibody expression vectors. This approach was applied to antibodies 122G3, 32H2, 34G9, and 102G10 resulting in the identification of the complete antibody sequence of the Ig-variable region as it occurred in the patient. This sequence was used for recombinant production of these antibodies which were then used in the subsequent characterization steps.

### Antibody Production and Purification

Transient gene expression of human antibodies was achieved upon transfection of antibody expression vectors into 293-T human embryonic kidney cells or Chinese Hamster Ovary cells (CHO using the Polyethylenimine Transfection method (PEI, Polyscience Warrington, USA). After transfection cells were cultured in serum free medium (OPTI-MEM I supplemented with GlutaMAX-I Gibco). Supernatants were collected after 3-6 days of culture and IgG was purified using protein A columns (GE HealthCare, Sweden) on a fast protein liquid chromatography device (FPLC) (GE HealthCare, Sweden).

### Experiment 2

### Epitope Mapping of Antibodies

### Epitope Mapping ELISA

20mer peptides spanning the entire MAGEA3 protein with 10 aa overlaps shared by each adjacent peptide (Peptides&Elephants, Nuthetal, Germany) were used to coat Maxisorp ELISA plates (Nunc, Rochester, N.Y.). As control a 20mer peptide representing amino acids 40-21 from the NY-ESO-1 protein was used (rev). The amino acids of MAGEA3 represented by the 20mer peptides are indicated in the table below. The numbering of amino acids of MAGEA3 is based on SEQ ID No:41.

| | MAGEA3 PEPTIDE No | | | | | | | |
|---|---|---|---|---|---|---|---|---|
| | 1 | 2 | 3 | 4 | 5 | 6 | 7 | 8 |
| Amino acid | 1-20 | 11-30 | 21-40 | 31-50 | 41-60 | 51-70 | 61-80 | 71-90 |
| | MAGEA3 PEPTIDE No | | | | | | | |
| | 17 | 18 | 19 | 20 | 21 | 22 | 23 | 24 |
| Amino acid | 161-180 | 171-190 | 181-200 | 191-210 | 201-220 | 211-230 | 221-240 | 231-250 |
| | MAGEA3 PEPTIDE No | | | | | | | |
| | 9 | 10 | 11 | 12 | 13 | 14 | 15 | 16 |
| Amino acid | 81-100 | 91-110 | 101-120 | 111-130 | 121-140 | 131-150 | 141-160 | 151-170 |
| | MAGEA3 PEPTIDE No | | | | | | | |
| | 25 | 26 | 27 | 28 | 29 | 30 | 31 | rev |
| Amino acid | 241-260 | 251-270 | 261-280 | 271-290 | 281-300 | 291-310 | 295-314 | NY- |
| | | | | | | | | ESO-1 |
| | | | | | | | | aa 40- |
| | | | | | | | | 21 |

Human recombinant antibody was used at a concentration of 1 μg/ml. Bound human antibody was detected using horseradish peroxidase-conjugated goat anti-human IgG Fc-gamma specific antibodies (Jackson ImmunoResearch, Europe Ltd., Cambridgeshire, UK).

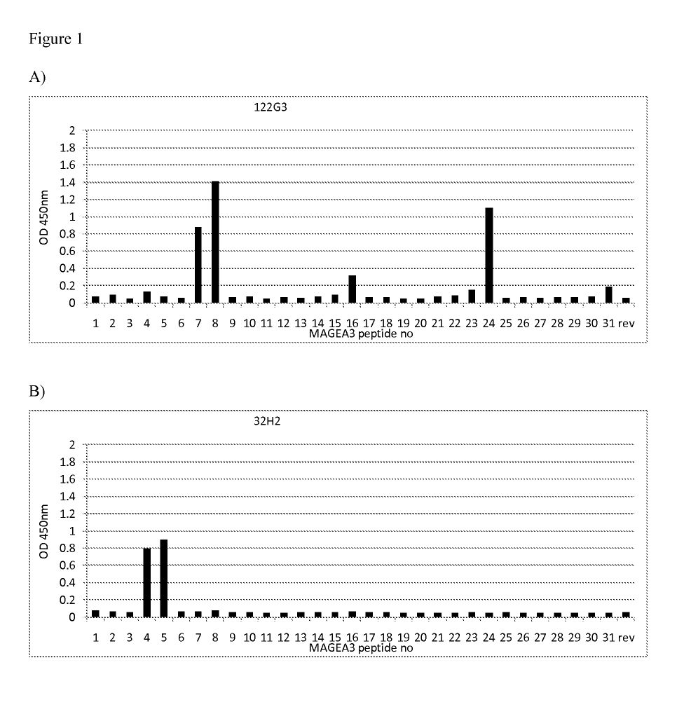

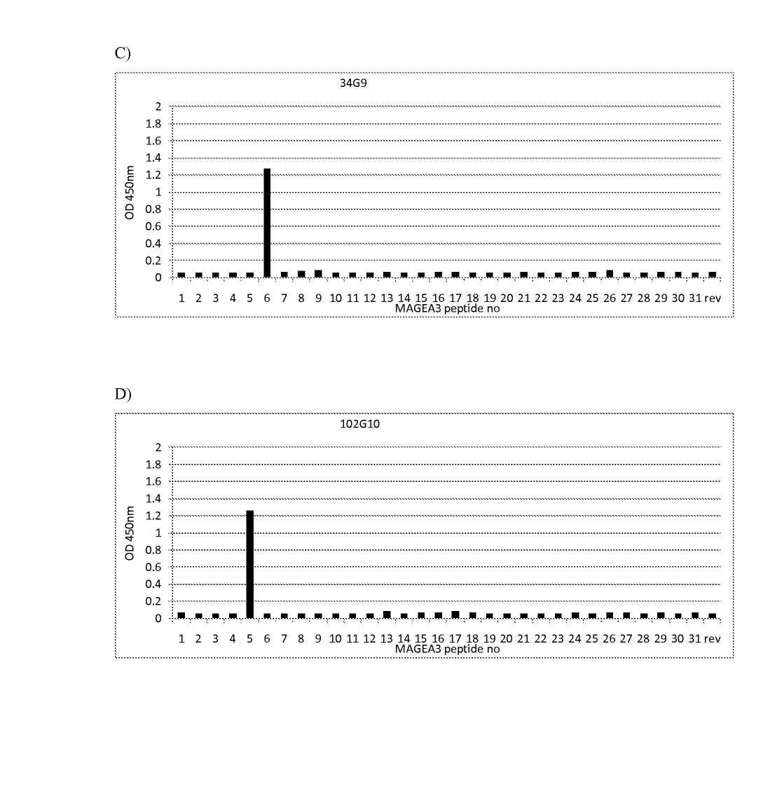

The results are depicted in FIG. 1

### Experiment 3

### Immunoprecipitation of MAGEA3

Purified recombinant MAGEA3 protein and lysate prepared from SK− Mel-37 cells known to endogenously express MAGEA3, were incubated with human MAGEA3-specific antibody followed by incubation with Protein G beads (New England Biolabs, Bioconcept, Allschwil, Switzerland). Bound protein was analyzed by Western Blot after Gradient SDS Polyacrylamide Gel Electrophoresis (NuPage 4-12% Bis-Tris Gel, Invitrogen, LuBioScience, Lucerne, Switzerland). As detection antibody the mouse monoclonal antibody M3H67 was used (provided by LICR, New York, USA).

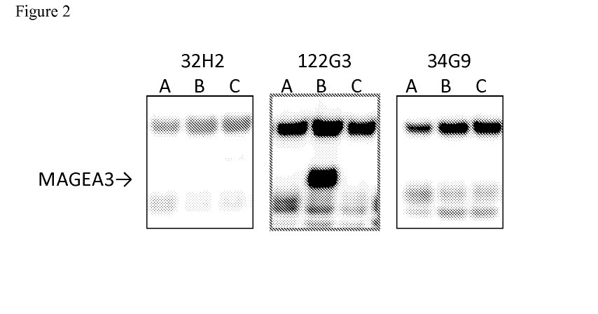

The results are depicted in FIG. 2.

### Experiment 4

### Differential Binding to MAGEA3 and MAGEA4

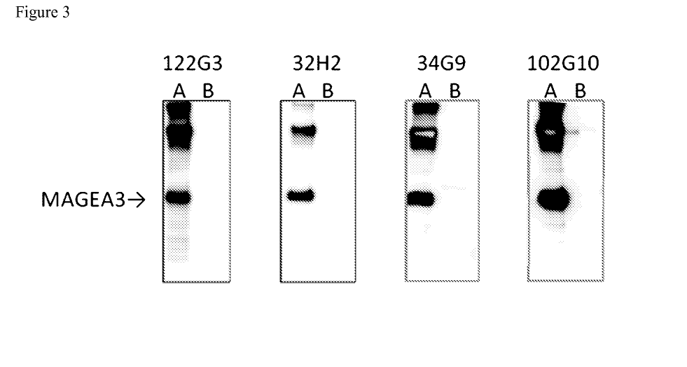

Recombinant MAGEA3- and recombinant MAGEA4-protein were separated in SDS-PAGE and blotted onto membranes. Membranes were incubated with human antibodies 122G3 or 32H2. Binding of human antibodies to proteins on the membrane was evaluated using an HRP-labeled goat anti human IgG (Jackson Immunoresearch, Milan Analytica AG, Rheinfelden, Switzerland). The results are depicted in FIG. 3.

### Experiment 5

### EC50 Determination of MAGEA3 Antibodies

Saturation experiments identified the half-maximal binding concentration of human monoclonal antibodies 122g3, 32H2, 34G9 and 122G10 to their corresponding MAGEA3 peptides. In competition experiments, increasing concentrations of MAGEA3 peptides ranging from 10 pM to 100 μM were mixed with their appropriate antibody at a concentration of 1.5 ng/ml and the mix was then transferred to an ELISA plate coated with the corresponding peptide at 10 mg/ml.

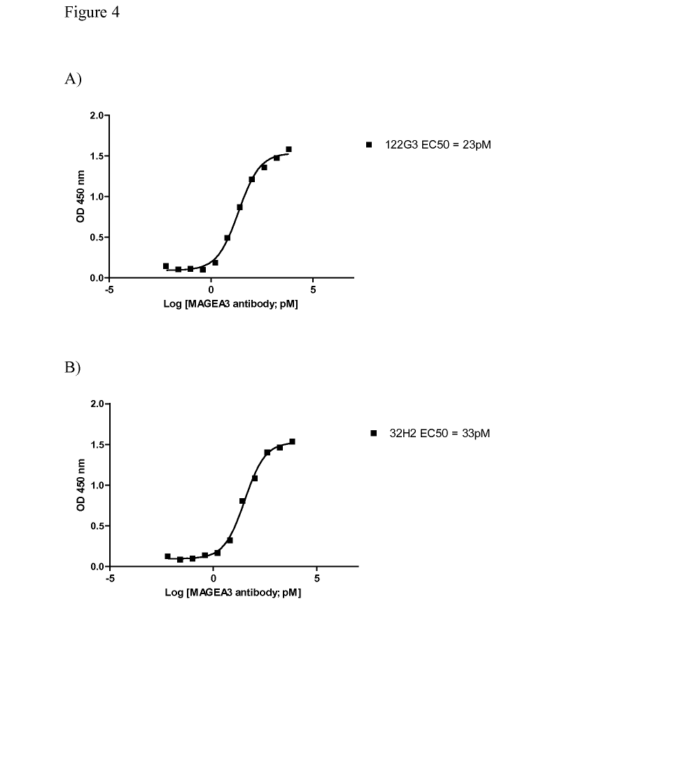

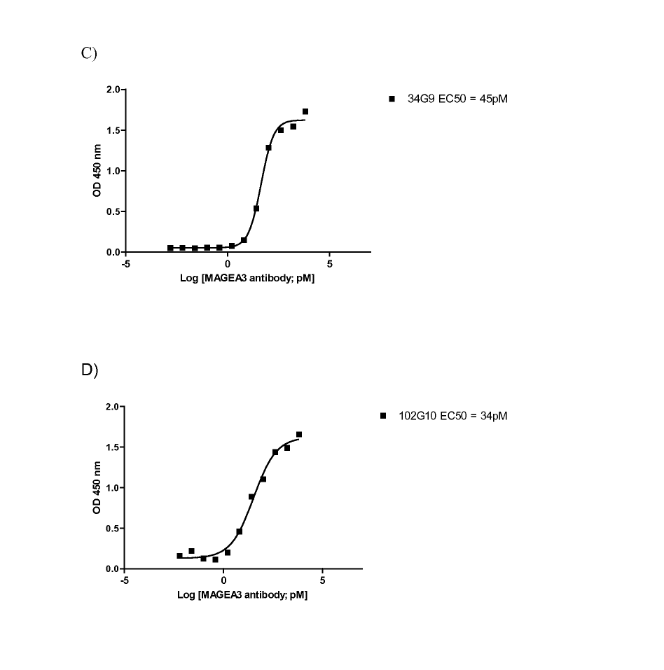

The results are depicted in FIG. 4

### Experiment 6

### Antibody Specificity

Recombinantly expressed MAGEA3, MAGEA6, MAGEA2, MAGEA1, MAGEA4, MAGEA10, MAGE-C1, MAGE-C2 and DHFR as negative control were coated on ELISA plates and binding of MAGEA3 antibodies 122G3, and 32H2 to these proteins was tested.

The results are summarized in the below table

| Antigen | 122G3 | 32H2 |
|---|---|---|
| MAGEA3 | +++ | +++ |
| MAGEA1 | − | − |
| MAGEA4 | − | − |
| MAGEA10 | − | − |
| MAGE-C1 | − | − |
| MAGE-C2 | − | − |
| DHFR | − | − |

### Experiment 7

### T Cell Stimulation

### Monocyte-derived DC

Monocytes are isolated from PBMC obtained from a voluntary donor using anti CD14 antibodies coupled to magnetic particles (MACS, Miltenyi Biosciences, Bergisch Gladbach, Germany). Monocytes are cultured at a cell density of 2×10Exp6/ml in DC medium (CellGro DC media, CellGenix Freiburg, Germany) supplemented with GM-CSF and IL-4 (Peprotech, London, UK). On day 5 Monocyte-derived DC are harvested and incubated with immune complexes in a 96-well flat-bottom plate. Maturation is induced by the addition of TNF-alpha and sCD40L.

### Immune Complex Formation

Recombinant MAGEA3 protein is incubated with the various human monoclonal antibodies at an equimolar ratio in CellGro medium.

### T Cell Stimulation Assay

Matured Monocyte-derived DC co-incubated or not with immune complexes and MAGEA3 specific CD8 T cells corresponding are incubated in microtiter plates with a 1:1 cellular ratio in RPMI supplemented with human serum and Brefeldin A.

Production of intracellular IFN-gamma is then monitored after permeabilising and fixing the cells using intra cellular staining with fluorescently labeled antibodies anti-IFN-gamma

### Experiment 8

### Antibody-Mediated Anti-Tumor Effects In Vivo

Mice are inoculated with syngeneic tumor cells expressing MAGEA3 and cytotoxic therapy is applied once the tumors are palpable. Subsequently, MAGEA3-specific antibody is administered. Effects of treatment on tumor growth are measured by monitoring tumor area over time using a caliper.

Induction and/or enhancement of immune effector cell activity against the tumor will be measured by analysis of tumor infiltrating lymphocytes, ex vivo CTL-assays, ex vivo cytokine secretion, in vivo CTL-assays or by monitoring shifts in immune effector cell repertoire subsequent to administration of MAGEA3-specific antibody in comparison to controls.

Some embodiments of the invention relate to:

1. Isolated monoclonal MAGEA3-binding antibody or binding fragment thereof.

2. Isolated monoclonal MAGEA3-binding antibody or binding fragment thereof according to embodiment 1., which binds preferentially to MAGE_A3.

3. Isolated monoclonal MAGEA3-binding antibody or binding fragment thereof according to embodiment 1. or 2., which binds to MAGEA3 but not to MAGEA4.

4. Isolated monoclonal MAGEA3-binding antibody or binding fragment thereof according to any of embodiments 1., 2. or 3., which binds to MAGEA3 but not to MAGEA4, MAGEA1 and/or MAGEA10.

5. Isolated monoclonal MAGEA3-binding antibody or binding fragment thereof according to any of embodiments 1., 2., 3., or 4., which binds to MAGEA3 but not to MAGEA2.

6. Isolated monoclonal MAGEA3-binding antibody or binding fragment thereof according to any of embodiments 1., 2., 3., 4., or 5., which binds to MAGEA3 but not to MAGEA6.

7. Isolated monoclonal MAGEA3-binding antibody or binding fragment thereof according to any of embodiments 1., 2., 3., 4., 5. or 6., which binds to MAGEA3 with a KD of about 300 pM or less.

8. Isolated monoclonal MAGEA3-binding antibody or binding fragment thereof according to embodiment 7., which binds to MAGEA3 with a K_{D} of about 200 pM or less.

9. Isolated monoclonal MAGEA3-binding antibody or binding fragment thereof according to embodiment 8., which binds to MAGEA3 with a K_{D} of about 100 pM or less.

10. Isolated monoclonal MAGEA3-binding antibody or binding fragment thereof according to embodiment 9., which binds to MAGEA3 with a K_{D} of about 50 pM or less.

11. Isolated monoclonal MAGEA3-binding antibody or binding fragment thereof according to any of embodiments 1., 2., 3., 4., 5., 6., 7., 8., 9., or 10., which binds to an epitope comprising SEQ ID No. 42.

12. Isolated monoclonal MAGEA3-binding antibody or binding fragment thereof according to any of embodiments 1., 2., 3., 4., 5., 6., 7., 8., 9., or 10., which binds to an epitope comprising SEQ ID No. 43.

13. Isolated monoclonal MAGEA3-binding antibody or binding fragment thereof according to any of embodiments 1., 2., 3., 4., 5., 6., 7., 8., 9., or 10., which binds to an epitope comprising SEQ ID No. 44.

14. Isolated monoclonal MAGEA3-binding antibody or binding fragment thereof according to any of embodiments 1., 2., 3., 4., 5., 6., 7., 8., 9., 10., 11., 12., or 13., which comprises a light chain variable region and/or a heavy chain variable region, wherein
a. the light chain variable region comprises at least a CDR1 selected from SEQ ID Nos.: 8, 18, 28, 38 or sequences at least 80% identical thereto, a CDR2 selected from SEQ ID Nos.: 9, 19, 19, 29, 39 or sequences at least 80% identical thereto, and/or a CDR3 selected from SEQ ID Nos.: 10, 20, 30, 40 or sequences at least 80% identical thereto; and/or wherein
b. the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and/or a CDR3 selected from SEQ ID Nos.: 7, 17, 27, 37 or sequences at least 80% identical thereto.

15. Isolated monoclonal MAGEA3-binding antibody or binding fragment thereof according to embodiment 14., which comprises a light chain variable region and/or a heavy chain variable region, wherein
a. the light chain variable region comprises at least a CDR1 selected from SEQ ID Nos.: 8, 18, 28, 38 or sequences at least 80% identical thereto, a CDR2 selected from SEQ ID Nos.: 9, 19, 19, 29, 39 or sequences at least 80% identical thereto, and a CDR3 selected from SEQ ID Nos.: 10, 20, 30, 40 or sequences at least 80% identical thereto; and/or wherein
b. the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and a CDR3 selected from SEQ ID Nos.: 7, 17, 27, 37 or sequences at least 80% identical thereto.

16. Isolated monoclonal MAGEA3-binding antibody or binding fragment thereof according to any of embodiments 1., 2., 3., 4., 5., 6., 7., 8., 9., 10., 11., 12., or 13., which comprises a light chain variable region comprising SEQ ID Nos.: 4, 14, 24, 34 or sequences at least 80% identical thereto and/or a heavy chain variable region comprising SEQ ID Nos.: 3, 13, 23, 33 or sequences at least 80% identical thereto.

17. Isolated monoclonal MAGEA3-binding antibody or binding fragment thereof according to embodiment 16., which comprises a light chain variable region comprising SEQ ID Nos.: 4, 14, 24, 34 or sequences at least 80% identical thereto and a heavy chain variable region comprising SEQ ID Nos.: 3, 13, 23, 33 or sequences at least 80% identical thereto.

18. Isolated monoclonal MAGEA3-binding antibody or binding fragment thereof which comprises a light chain variable region and/or a heavy chain variable region, wherein
a. the light chain variable region comprises at least a CDR1 selected from SEQ ID Nos.: 8, 18, 28, 38 or sequences at least 80% identical thereto, a CDR2 selected from SEQ ID Nos.: 9, 19, 19, 29, 39 or sequences at least 80% identical thereto, and/or a CDR3 selected from SEQ ID Nos.: 10, 20, 30, 40 or sequences at least 80% identical thereto; and/or wherein
b. the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and/or a CDR3 selected from SEQ ID Nos.: 7, 17, 27, 37 or sequences at least 80% identical thereto.

19. Isolated monoclonal MAGEA3-binding antibody or binding fragment thereof according to embodiment 18., which comprises a light chain variable region and/or a heavy chain variable region, wherein
a. the light chain variable region comprises at least a CDR1 selected from SEQ ID Nos.: 8, 18, 28, 38 or sequences at least 80% identical thereto, a CDR2 selected from SEQ ID Nos.: 9, 19, 19, 29, 39 or sequences at least 80% identical thereto, and a CDR3 selected from SEQ ID Nos.: 10, 20, 30, 40 or sequences at least 80% identical thereto; and/or wherein
b. the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and a CDR3 selected from SEQ ID Nos.: 7, 17, 27, 37 or sequences at least 80% identical thereto.

20. Isolated monoclonal MAGEA3-binding antibody or binding fragment thereof according to embodiment 19., which comprises a light chain variable region and/or a heavy chain variable region, wherein
a. the light chain variable region comprises at least a CDR1 selected from SEQ ID Nos.: 8, 18 or sequences at least 80% identical thereto, a CDR2 selected from SEQ ID Nos.: 9, 19 or sequences at least 80% identical thereto, and a CDR3 selected from SEQ ID Nos.: 10, 20 or sequences at least 80% identical thereto; and/or wherein
b. the heavy chain variable region comprises at least a CDR1 selected from SEQ ID Nos.: 5, 15 or sequences at least 80% identical thereto, a CDR2 selected from SEQ ID Nos.: 6, 16 or sequences at least 80% identical thereto, and a CDR3 selected from SEQ ID Nos.: 7, 17 or sequences at least 80% identical thereto.

21. Isolated monoclonal MAGEA3-binding antibody or binding fragment thereof, which comprises a light chain variable region comprising SEQ ID Nos.: 4, 14, 24, 34 or sequences at least 80% identical thereto and/or a heavy chain variable region comprising SEQ ID Nos.: 3, 13, 23, 33 or sequences at least 80% identical thereto.

22. Isolated monoclonal MAGEA3-binding antibody or binding fragment according to embodiment 21, which comprises a light chain variable region comprising SEQ ID Nos.: 4, 14, 24, 34 or sequences at least 80% identical thereto and a heavy chain variable region comprising SEQ ID Nos.: 3, 13, 23, 33 or sequences at least 80% identical thereto.

23. Isolated monoclonal MAGEA3-binding antibody or binding fragment according to embodiment 22, which comprises a light chain variable region comprising SEQ ID Nos.: 4, 14 or sequences at least 80% identical thereto and a heavy chain variable region comprising SEQ ID Nos.: 3, 13 or sequences at least 80% identical thereto.

24. Nucleic acid molecule comprising a nucleic acid sequence coding for a variable heavy chain of SEQ ID No.: 3, 13, 23, 33 or a sequence 80% identical thereto.

25. Nucleic acid molecule comprising a nucleic acid sequence coding for a variable light chain of SEQ ID No.: 4, 14, 24, 34 or a sequence 80% identical thereto.

26. Nucleic acid molecule comprising a nucleic acid sequence coding for a variable heavy chain region CRD of SEQ ID Nos.: 5, 15, 25, 35, 6, 16, 26, 36, 7, 17, 27, 37 or a sequence at least 80% identical thereto.

27. Nucleic acid molecule comprising a nucleic acid sequence coding for a variable light chain region CRD of SEQ ID Nos.: 8, 18, 28, 38, 9, 19, 29, 39, 10, 20, 30, 40 or a sequence 80% identical thereto.

28. A vector comprising a nucleic acid molecule according to any of embodiments 24 to 27.

29. A cell being transformed with a nucleic acid molecule according to any of embodiments 24 to 27 or a vector or embodiment 28.

30. Pharmaceutical composition comprising a MAGEA3-binding antibody or binding fragment thereof in accordance with any of embodiments 1 to 23, a nucleic acid molecule in accordance with any of embodiments 24 to 27, a vector in accordance with embodiment 28 or a cell in accordance with embodiment 29.

31. Pharmaceutical composition in accordance with embodiment 30, which does not comprise a compound capable of activating the immune system.

32. Pharmaceutical composition in accordance with embodiment 30 comprising said MAGEA3-binding antibody or binding fragment thereof as the sole pharmaceutically active agent.

33. Pharmaceutical composition in accordance with any of embodiments 30 to 32 for use in the treatment of a hyper-proliferative disease.

34. Use of antibody or binding fragment thereof in accordance with any of embodiments 1 to 23, a nucleic acid molecule in accordance with any of embodiments 24 to 27, a vector in accordance with embodiment 28, a cell in accordance with embodiment 29 or a pharmaceutical composition in accordance with any of claims 31 to 32 in the manufacture of a medicament for treating a hyper-proliferative disease.

35. Method of treating a hyper-proliferative disease by administering to a patient in need thereof an antibody or binding fragment thereof in accordance with any of embodiments 1 to 23 or a pharmaceutical composition in accordance with any of embodiments 30 to 32.

36. Pharmaceutical composition, use or method of any of embodiments 33 to 35, wherein the hyper-proliferative disease is characterized by expression of MAGEA3.

37. Pharmaceutical composition, use or method of any of embodiments 33 to 36, wherein said hyper-proliferative disease is selected from basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer and Wilms tumor.

38. Pharmaceutical composition, use or method of any of embodiments 33 to 36, wherein said hyper-proliferative disease is selected from melanoma, breast cancer, ovarian cancer, non-small cell lung cancer, multiple myeloma and/or pancreatic cancer.

39. Pharmaceutical composition comprising at least one tumor associated antigen (TAA) binding antibody or binding fragment thereof and at least one compound capable of activating the immune system.

40. Kit of pharmaceutical compositions comprising
a) a first pharmaceutical composition comprising at least one tumor associated antigen (TAA) binding antibody or binding fragment thereof; and
b) a second pharmaceutical composition comprising at least one compound capable of activating the immune system.

41. Pharmaceutical composition according to embodiment 39 or kit according to embodiment 40, wherein the at least one TAA binding antibody or binding fragment thereof binds to a CT antigen.

42. Pharmaceutical composition or kit according to any of embodiments 39 to 41, wherein the at least one TAA binding antibody or binding fragment thereof binds to a CT antigen selected from table 1.

43. Pharmaceutical composition or kit according to any of embodiments 39 to 42, wherein the at least one TAA binding antibody or binding fragment thereof is a monoclonal chimeric, humanized or human antibody or binding fragment thereof.

44. Pharmaceutical composition or kit according to any of embodiments 39 to 43, wherein the at least one TAA binding antibody or binding fragment thereof is a monoclonal human patient-derived antibody or binding fragment thereof.

45. Pharmaceutical composition or kit according to any of embodiments 39 to 44, wherein the at least one TAA binding antibody or binding fragment thereof comprises a constant region selected from the IgG class.

46. Pharmaceutical composition or kit according to any of embodiments 39 to 45, wherein the at least one TAA binding antibody or binding fragment thereof binds to the TAA with a Kd of about 0.1*10⁻¹² to about 1*10⁻⁶M.

47. Pharmaceutical composition or kit according to any of embodiments 39 to 46 wherein the TAA-antibody or binding fragment thereof and/or any other antibody or binding fragment thereof which is part of the pharmaceutical compositions or kits in accordance with any of embodiments 39 to 46 is coupled to a drug, a radioisotope, lectins, and/or a toxin.

48. Pharmaceutical composition or kit according to any of embodiments 39 to 47, wherein the at least one TAA binding antibody or binding fragment thereof binds to MAGEA3.

49. Pharmaceutical composition or kit according to any of embodiments 39 to 48, wherein the at least one TAA binding antibody or binding fragments thereof binds to MAGEA3 and is a patient-derived monoclonal human antibody or binding fragment thereof.

50. Pharmaceutical composition or kit according to any of embodiments 39 to 49, wherein the at least one TAA binding antibody or binding fragments thereof binds to MAGEA3 and comprises a light chain variable region and/or a heavy chain variable region, wherein
a. the light chain variable region comprises at least a CDR1 selected from SEQ ID Nos.: 8, 18, 28, 38 or sequences at least 80% identical thereto, a CDR2 selected from SEQ ID Nos.: 9, 19, 29, 39 or sequences at least 80% identical thereto, and/or a CDR3 selected from SEQ ID Nos.: 10, 20, 30, 40 or sequences at least 80% identical thereto; and/or wherein
b. the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and/or a CDR3 selected from SEQ ID Nos.: 7, 17, 27, 37 or sequences at least 80% identical thereto.

51. Pharmaceutical composition or kit according to embodiment 50, wherein the at least one TAA binding antibody or binding fragments thereof binds to MAGEA3 and comprises a light chain variable region and/or a heavy chain variable region, wherein
a. the light chain variable region comprises at least a CDR1 selected from SEQ ID Nos.: 8, 18, 28, 38 or sequences at least 80% identical thereto, a CDR2 selected from SEQ ID Nos.: 9, 19, 29, 39 or sequences at least 80% identical thereto, and a CDR3 selected from SEQ ID Nos.: 10, 20, 30, 40 or sequences at least 80% identical thereto; and/or wherein
b. the heavy chain variable region comprises at least a CDR1 selected from SEQ ID Nos.: 5, 15, 25, 35 or sequences at least 80% identical thereto, a CDR2 selected from SEQ ID Nos.: 6, 16, 26, 36 or sequences at least 80% identical thereto, and a CDR3 selected from SEQ ID Nos.: 7, 17, 27, 37 or sequences at least 80% identical thereto.

52. Pharmaceutical composition or kit according to any of embodiments 39 to 49, wherein the at least one TAA binding antibody or binding fragment thereof binds to MAGEA3 and wherein the antibody or binding fragment comprises a light chain variable region comprising SEQ ID Nos.: 4, 14, 24, 34 or sequences at least 80% identical thereto and/or a heavy chain variable region comprising SEQ ID Nos.: 3, 13, 23, 33 or sequences at least 80% identical thereto.

53. Pharmaceutical composition or kit according to embodiment 52, wherein the at least one TAA binding antibody or binding fragment thereof binds to MAGEA3 and wherein the antibody or binding fragment comprises a light chain variable region comprising SEQ ID Nos.: 4, 14, 24, 34 or sequences at least 80% identical thereto and/or a heavy chain variable region comprising SEQ ID Nos.: 3, 13, 23, 33 or sequences at least 80% identical thereto.

54. Pharmaceutical composition or kit according to any of embodiments 39 to 53, wherein the at least one compound capable of activating the immune system is selected from natural stimulants or at least co-stimulants of the immune system, agonistic activators of natural stimulants or at least co-stimulants of the immune system, or antagonistic effectors of natural inhibitors or at least co-inhibitors of the immune system.

55. Pharmaceutical composition or kit according to any of embodiments 39 to 54, wherein the at least one compound capable of activating the immune system is selected from CD40L, anti-CD40 agonistic antibodies, anti-OX40 agonistic antibodies, anti-CD137 agonistic antibodies, anti-CTLA4 antagonistic antibodies, and anti-CD25 antagonistic antibodies.

56. Pharmaceutical composition or kit according to any of embodiments 39 to 55, wherein the at least one compound capable of activating the immune system is selected from CD40L, CP-870,893, SGN-40, Tremelimumab and Ipilimumab.

57. Pharmaceutical composition or kit according to any of embodiments 39 to 56, wherein the composition or the kit comprises at least two compounds capable of activating the immune system, of which the first compound is selected from natural stimulants or at least co-stimulants of the immune system or agonistic activators of natural stimulants or at least co-stimulants of the immune system, and of which the second compound is selected from antagonistic effectors of natural inhibitors or at least co-inhibitors of the immune system.

58. Pharmaceutical composition or kit according to embodiment 57, wherein the first compound capable of activating the immune system is selected from CD40L, anti-CD40 agonistic antibodies, anti-OX40 agonistic antibodies and anti-CD137 agonistic antibodies and wherein the second compound capable of activating the immune system is selected from anti-CTLA4 antagonistic antibodies, and anti-CD25 antagonistic antibodies.

59. Pharmaceutical composition or kit according to embodiments 58, wherein the first compound capable of activating the immune system is selected from CD40L, CP-870,893 and SGN-40, and wherein the second compound capable of activating the immune system is selected from Tremelimumab and Ipilimumab.

60. Pharmaceutical composition according to embodiment 39 or any of embodiments 41 to 59, wherein the at least one TAA binding antibody or binding fragment thereof and the at least one compound capable of activating the immune system take the form of a bi-specific antibody or binding fragment thereof.

61. Pharmaceutical composition according to embodiment 60, wherein the bi-specific antibody comprises (i) a TAA binding portion and (ii) a portion acting as agonistic activator of natural stimulants or at least co-stimulants of the immune system, or antagonistic effector of natural inhibitors or at least co-inhibitors of the immune system.

62. Pharmaceutical composition according to embodiment 61, wherein the bi-specific antibody comprises (i) a CT-antigen binding portion and (ii) a portion acting as anti-CD40 agonistic antibody, anti-OX40 agonistic antibody, anti-CD137 agonistic antibody, anti-CTLA4 antagonistic antibody, or anti-CD25 antagonistic antibody.

63. Pharmaceutical composition according to embodiment 62, wherein the bi-specific antibody comprises (i) a MAGEA3 binding portion and (ii) a portion acting as anti-CD40 agonistic antibody or anti-CTLA4 antagonistic antibody.

64. Pharmaceutical composition according to any of embodiments 39 or 41 to 63, wherein the composition comprises additionally a cytotoxic agent.

65. Pharmaceutical composition according to embodiment 39 or any of embodiments 41 to 64, wherein the cytotoxic agent is selected from 5-fluoro-uracil, taxanes, anthracyclines, cisplatin, carboplatin, gemcitabine, capecitabin, navelbine or zoledronate.

66. Kit according to any of embodiments 40 to 59, wherein the kit comprises a third pharmaceutical composition comprising a cytotoxic agent.

67. Kit according to embodiment 66, wherein the cytotoxic agent is selected from 5-fluoro-uracil, taxanes, anthracyclines, cisplatin, carboplatin, gemcitabine, capecitabin, navelbine or zoledronate.

68. Pharmaceutical composition or kit according to any of embodiments 39 to 67 comprising a cytotoxic agent, a CT-antigen binding antibody or binding fragment thereof, and at least one compound selected from (i) natural stimulants or at least co-stimulants of the immune system, (ii) agonistic activators of natural stimulants or at least co-stimulants of the immune system and/or (iii) antagonistic effectors of natural inhibitors or at least co-inhibitors of the immune system.

69. Pharmaceutical composition or kit according to any of embodiments 39 to 68 comprising a cytotoxic agent, a CT-antigen binding antibody or binding fragment thereof, and at least one compound selected from agonistic activators of natural stimulants or at least co-stimulants of the immune system, or from antagonistic effectors of natural inhibitors or at least co-inhibitors of the immune system.

70. Pharmaceutical composition or kit according to any of embodiments 39 to 69 comprising a cytotoxic agent, a CT-antigen binding antibody or binding fragment thereof, at least one compound selected from agonistic activators of natural stimulants or at least co-stimulants of the immune system, and at least one compound selected from antagonistic effectors of natural inhibitors or at least co-inhibitors of the immune system.

71. Pharmaceutical composition or kit according to any of embodiments 68 to 70, wherein the CT-antigen binding antibody or binding fragments thereof recognizes MAGEA3, wherein the at least one compound selected from agonistic activators of natural stimulants or at least co-stimulants of the immune system is an anti-CD40 agonistic antibody and wherein the at least one compound selected from antagonistic effectors of natural inhibitors or at least co-inhibitors of the immune system is an anti-CTLA4 antagonistic antibody.

72. Combination of at least one tumor associated antigen (TAA) binding antibody or binding fragment thereof and at least one compound capable of activating the immune system for use in treating a patient wherein a TAA binding antibody or binding fragment thereof and at least one compound capable of activating the immune system is administered to the patient.

73. Combination for use as in embodiment 72, wherein a TAA binding antibody or binding fragment thereof and at least one compound capable of activating the immune system as mentioned in any of embodiments 41 to 63 is administered to the patient.

74. Combination for use as in embodiment 73, wherein the patient is subjected to cytotoxic treatment prior to, simultaneous with or subsequent to administration of said combination.

75. Combination for use as in embodiment 74, wherein the cytotoxic treatment includes chemotherapy, radiation therapy, surgery and/or hyperthermia.

76. Combination for use as in embodiment 75, wherein chemotherapy includes administration of agents selected from 5-fluoro-uracil, taxanes, anthracyclines, cisplatin, carboplatin, gemcitabine, capecitabin, navelbine or zoledronate.

77. Combination for use as in embodiments 72 to 76 for treating a hyper-proliferative disease.

78. Combination for use as in embodiment 77 for treating a hyper-proliferative disease, which is characterized by expression of a TAA.

79. Combination for use as in embodiment 78, wherein said TAA is a CT antigen.

80. Combination for use as in embodiment 79, wherein said CT antigen is MAGEA3.

81. Combination for use as in any of embodiments 72 to 80, wherein said hyper-proliferative disease is selected from basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer; and Wilms tumor.

82. Medicament for use in treating a patient wherein a pharmaceutical composition or a kit in accordance with any of embodiments 39 to 71 or a combination of at least one tumor associated antigen (TAA) binding antibody or binding fragment thereof and at least one compound capable of activating the immune system is administered to the patient.

83. Medicament for use as in embodiment 82, wherein the patient is subjected to cytotoxic treatment prior to, simultaneous with or subsequent to administration of a pharmaceutical composition or a kit in accordance with any of embodiments 39 to 71 or of a combination of at least one tumor associated antigen (TAA) binding antibody or binding fragment thereof and at least one compound capable of activating the immune system.

84. Medicament for use as in embodiment 83, wherein the cytotoxic treatment includes chemotherapy, radiation therapy, surgery and/or hyperthermia.

85. Medicament for use as in embodiment 84, wherein chemotherapy includes administration of agents selected from 5-fluoro-uracil, taxanes, anthracyclines, cisplatin, carboplatin, gemcitabine, capecitabin, navelbine or zoledronate.

86. Medicament for use as in embodiments 82 to 85 for treating a hyper-proliferative disease.

87. Medicament for use as in embodiment 86 for treating a hyper-proliferative disease, which is characterized by expression of a TAA.

88. Medicament for use as in embodiment 87, wherein said TAA is a CT antigen.

89. Medicament for use as in embodiment 88, wherein said CT antigen is MAGEA3.

90. Medicament for use as in any of embodiments 86 to 89, wherein said hyper-proliferative disease is selected from basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer and Wilms tumor.

91. Medicament for use as in any of embodiments 82 to 90, wherein the at least one tumor associated antigen (TAA) binding antibody or binding fragment thereof and at least one compound capable of activating the immune system are as mentioned in any of embodiments 39 to 71.

92. Use of a pharmaceutical composition or a kit in accordance with any of embodiments 39 to 71 or a combination of at least one tumor associated antigen (TAA) binding antibody or binding fragment thereof and at least one compound capable of activating the immune system in the manufacture of a medicament for treating a patient.

93. Use as in embodiment 92, wherein the patient is subjected to cytotoxic treatment prior to, simultaneous with or subsequent to the administration of a pharmaceutical composition or a kit in accordance with any of embodiments 39 to 71 or of a combination of at least one tumor associated antigen (TAA) binding antibody or binding fragment thereof and at least one compound capable of activating the immune system.

94. Use as in embodiment 93, wherein the cytotoxic treatment includes chemotherapy, radiation therapy, surgery and/or hyperthermia.

95. Use as in embodiment 94, wherein chemotherapy includes administration of agents selected from 5-fluoro-uracil, taxanes, anthracyclines, cisplatin, carboplatin, gemcitabine, capecitabin, navelbine or zoledronate.

96. Use as in embodiments 92 to 95 for treating a hyper-proliferative disease.

97. Use as in embodiment 96 for treating a hyper-proliferative disease, which is characterized by expression of a TAA.

98. Use as in embodiment 97, wherein said TAA is a CT antigen.

99. Use as in embodiment 98, wherein said CT antigen is MAGEA3.

100. Use as in any of embodiments 92 to 99, wherein said hyper-proliferative disease is selected from basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer and Wilms tumor.

101. Use as in any of embodiments 92 to 100, wherein the at least one tumor associated antigen (TAA) binding antibody or binding fragment thereof and at least one compound capable of activating the immune system are as mentioned in any of embodiments 41 to 63.

102. Method of treating a patient by administering a pharmaceutical composition or a kit in accordance with any of embodiments 39 to 71 or a combination of at least one tumor associated antigen (TAA) binding antibody or binding fragment thereof and at least one compound capable of activating the immune system is administered to the patient.

103. Method as in embodiment 102, wherein the patient is subjected to cytotoxic treatment prior to, simultaneous with or subsequent to the administration of a pharmaceutical composition or a kit in accordance with any of embodiments 39 to 71 or of a combination of at least one tumor associated antigen (TAA) binding antibody or binding fragment thereof and at least one compound capable of activating the immune system.

104. Method as in embodiment 103, wherein the cytotoxic treatment includes chemotherapy, radiation therapy, surgery and/or hyperthermia.

105. Method as in embodiment 104, wherein chemotherapy includes administration of agents selected from 5-fluoro-uracil, taxanes, anthracyclines, cisplatin, carboplatin, gemcitabine, capecitabin, navelbine or zoledronate.

106. Method as in embodiments 102 to 105 for treating a hyper-proliferative disease.

107. Method as in embodiment 106 for treating a hyper-proliferative disease, which is characterized by expression of a TAA.

108. Method as in embodiment 107, wherein said TAA is a CT antigen.

109. Method as in embodiment 108, wherein said CT antigen is MAGEA3.

110. Method as in any of embodiments 102 to 109, wherein said hyper-proliferative disease is selected from basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer and Wilms tumor.

111. Method as in any of embodiments 102 to 110, wherein the at least one tumor associated antigen (TAA) binding antibody or binding fragment thereof and at least one compound capable of activating the immune system are as mentioned in any of embodiments 39 to 71.

112. MAGEA3-binding antibody or binding fragment thereof according to any of embodiments 1 to 23 for use in diagnosis.

113. Diagnostic composition comprising a MAGEA3-binding antibody or binding fragment thereof according to any of embodiments 1 to 23.

114. MAGEA3-binding antibody or binding fragment thereof for use as in embodiment 112 or diagnostic composition as in embodiment 113, wherein a hyperproliferative disease is diagnosed.

115. MAGEA3-binding antibody or binding fragment thereof for use or diagnostic composition as in embodiment 114, wherein the hyperproliferative disease is selected from basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer and Wilms tumor.

116. MAGEA3-binding antibody or binding fragment thereof for use or diagnostic composition as in embodiment 114 or 115, wherein the hyperproliferative disease is characterized by an overexpression of MAGEA3 and/or MAGEA6.

117. MAGEA3-binding antibody or binding fragment thereof for use or diagnostic composition as in embodiments 114 to 116, wherein said hyperproliferative disease is selected from melanoma, breast cancer, ovarian cancer, non-small cell lung cancer, multiple myeloma and/or pancreatic cancer.

118. A method of diagnosing a hyperproliferative disease in a human or animal individual comprising at least the steps of:
a. Testing a sample of said human or animal individual for expression of at least MAGEA3 by using an MAGEA3-binding antibody or binding fragment thereof according to any of embodiments 1 to 23;
b. Comparing with expression of at least MAGEA3 in a control sample;
c. Determining the occurrence and/or likely development of a hyperproliferative disease by comparing the data obtained in steps a) and b).

119. A method of data acquisition comprising at least the steps of:
a. Administering a MAGEA3 binding antibody or binding fragment thereof according to any of embodiments 1 to 23 to a human or animal individual;
b. Determining the distribution of said MAGEA3-binding antibody or binding fragment thereof in said human or animal individual; and
c. Determining expression of at least MAGEA3 in said human or animal individual by using a MAGEA3-binding antibody or binding fragment thereof according to any of embodiments 1 to 23.

120. Method according to embodiment 118 or 119, wherein the hyperproliferative disease is selected from basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer and Wilms tumor.

121. Method according to any of embodiments 118 to 120, wherein the hyperproliferative disease is characterized by an overexpression of MAGEA3 and/or MAGEA6.

122. Method according to any of embodiments 118 to 121, wherein said hyperproliferative disease is selected from melanoma, breast cancer, ovarian cancer, non-small cell lung cancer, multiple myeloma and/or pancreatic cancer.

123. Isolated monoclonal MAGEA3-binding antibody or binding fragment thereof according to any of embodiments 1., 2., 3., 4., 5., 6., 7., 8., 9., 10., 11., 12., 13., 14., 15., 16., 17., 18., 19., 20., 21., 22., or 23., wherein said antibody is a human monoclonal patient-derived MAGEA3 binding antibody or binding fragment thereof.

124. Pharmaceutical composition according to any of embodiments 30., 31., 32., 33., 36., 37., or 38., use according to any of embodiments 34., 36., 37., or 38., or method according to any of embodiments 35., 36., 37., or 38., wherein said antibody is a human monoclonal patient-derived MAGEA3 binding antibody or binding fragment thereof.

125. Pharmaceutical composition according to any of embodiments 39., 41., 42., 43., 44., 45., 46., 47., 48., 49., 50., 51., 52., 53., 54., 55., 56., 57., 58., 59., 60., 61., 62., 63., 64., 65., 68., 69., 70., or 71., or kit according to any of embodiments 40., 41., 42., 43., 44., 45., 46., 47., 48., 49., 50., 51., 52., 53., 54., 55., 56., 57., 58., 59., 66., 67., 68., 69., 70., or 71., wherein said antibody is a human monoclonal patient-derived MAGEA3 binding antibody or binding fragment thereof.

126. Combination according to any of embodiments 72., 73., 74., 75., 76., 77., 78., 79., 80., or 81., wherein said antibody is a human monoclonal patient-derived MAGEA3 binding antibody or binding fragment thereof.

127. Medicament according to any of embodiments 82., 83., 84., 85., 86., 87., 88., 89., 90., or 91., wherein said antibody is a human monoclonal patient-derived MAGEA3 binding antibody or binding fragment thereof.

128. Use according to any of embodiments 92., 93., 94., 95., 96., 97., 98., 99., 100., 101., wherein said antibody is a human monoclonal patient-derived MAGEA3 binding antibody or binding fragment thereof.

129. Method according to any of embodiments 102., 103., 104., 105., 106., 107., 108., 109., 110., or 111., wherein said antibody is a human monoclonal patient-derived MAGEA3 binding antibody or binding fragment thereof.

130. MAGEA3 binding antibody or binding fragment thereof for use in accordance with any of embodiments 112., 114., 115., 116., or 117., or diagnostic composition according to any of embodiments 113., 114., 115., 116., or 117., wherein said antibody is a human monoclonal patient-derived MAGEA3 binding antibody or binding fragment thereof.

131. Method according to any of embodiments 118., 119., 120., 121., or 122., wherein said antibody is a human monoclonal patient-derived MAGEA3 binding antibody or binding fragment thereof.

1-15. (canceled)

16. An isolated monoclonal antibody or binding fragment thereof that binds to MAGEA3, derived from a human patient vaccinated with MAGEA3.

17. The isolated monoclonal MAGEA3 binding antibody or binding fragment thereof according to claim 16 that comprises a light chain variable region and/or a heavy chain variable region, wherein
a. the light chain variable region comprises at least a CDR1 selected from SEQ ID NOs:8, 18, 28, 38 or sequences at least 80% identical thereto, a CDR2 selected from SEQ ID NOs:9, 19, 29, 39 or sequences at least 80% identical thereto, and a CDR3 selected from SEQ ID NOs:10, 20, 30, 40 or sequences at least 80% identical thereto, and/or wherein
b. the heavy chain variable region comprises at least a CDR1 selected from SEQ ID NOs:5, 15, 25, 35 or sequences at least 80% identical thereto, a CDR2 selected from SEQ ID NOs:6, 16, 26, 36 or sequences at least 80% identical thereto, and/or a CDR3 selected from SEQ ID NOs:7, 17, 27, 37 or sequences at least 80% identical thereto.

18. The isolated monoclonal MAGEA3 binding antibody or binding fragment thereof according to claim 16 that comprises a light chain variable region comprising SEQ ID NOs:4, 14, 24, 34 or sequences at least 80% identical thereto and a heavy chain variable region comprising SEQ ID NOs:3, 13, 23, 33 or sequences at least 80% identical thereto.

19. The isolated monoclonal MAGEA3 binding antibody or binding fragment thereof according to claim 16, which binds to an epitope comprising SEQ ID NO:42.

20. The isolated monoclonal MAGEA3 binding antibody or binding fragment thereof according to claim 16, which binds to an epitope comprising SEQ ID NO:45.

21. The isolated monoclonal MAGEA3 binding antibody or binding fragment thereof according to claim 16, which binds to an epitope comprising SEQ ID NO:46.

22. The isolated monoclonal MAGEA3 binding antibody or binding fragment thereof according to claim 16, which does not bind to MAGEA4, MAGEA1, and MAGEA10.

23. The isolated monoclonal MAGEA3 binding antibody or binding fragment thereof according to claim 16, which does not bind MAGEA2.

24. A pharmaceutical composition comprising the MAGEA3 binding antibody or binding fragment thereof according to claim 16.

25. A method of treatment of a hyper-proliferative disease comprising administration of the pharmaceutical composition comprising the MAGEA3 binding antibody or binding fragment thereof according to claim 24, wherein the hyper-proliferative disease is selected from the group consisting of basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer; and Wilms' tumor.

26. A method of treatment of a hyper-proliferative disease comprising administration of the MAGEA3 binding antibody or binding fragment thereof according to claim 16, wherein the hyper-proliferative disease is selected from the group consisting of basal cell carcinoma; bladder cancer; bone cancer; central nervous system tumors; Burkitt's lymphoma; breast cancer; cervical cancer; chronic myelogenous leukemia; colon cancer; rectal cancer; colorectal cancer, esophageal cancer; Ewing family of tumors; extrahepatic bile duct cancer; gallbladder cancer; gastrointestinal stromal tumor (GIST); glioma; head and neck cancer; islet cell tumors; Kaposi sarcoma; leukemia; liver cancer; lymphoma; Hodgkin's lymphoma; non-Hodgkin's lymphoma; mesothelioma; multiple myeloma/plasma cell neoplasm; myeloid leukemia; nasopharyngeal cancer; neuroblastoma; small cell lung cancer; non-small cell lung cancer; oropharyngeal cancer; ovarian cancer; pancreatic cancer; parathyroid cancer; penile cancer; pharyngeal cancer; phaeochromocytoma; pituitary tumor; prostate cancer; renal cell (kidney) cancer; respiratory tract carcinoma; retinoblastoma; skin cancer (melanoma); small intestine cancer; soft tissue sarcoma; squamous cell carcinoma; squamous neck cancer; stomach (gastric) cancer; T-cell lymphoma; testicular cancer; throat cancer; thyroid cancer; transitional cell cancer of the renal pelvis and ureter; urethral cancer; uterine cancer; vaginal cancer; vulvar cancer; and Wilms' tumor.

27. A diagnostic composition comprising the MAGEA3 binding antibody or binding fragment thereof according to claim 16.


SEQUENCE LISTING (50 sequences)

SEQ ID NO: 1 - DNA, 360 bp, Homo sapiens
  misc_feature: variable heavy chain - 122G3
caggtccaacttgtgcagtctggggctgaggtgaagaagcctggggcctcagtgaaggtttcctgcaaggcttctggatacaccttcactaactatgctatacattgggtgcgccaggcccccggacaaaggcttgagtggatgggatggatcaacgctggcaaaggtaacacaaaatattcacagaagttccagggcagagtcaccattaccagagacacatccgcggacacaacctacatggagctgagtagcctgagatctgaagacacggctgtgtatttctgtgcgcaagccgggcttagcagtggcttatacagggactactggggccagggaaccccggtcaccgtctcctca

SEQ ID NO: 2 - DNA, 321 bp, Homo sapiens
  misc_feature: variable light (kappa) chain - AB 122G3
gaaatagtgatgacgcagtctccagccaccctgtctgtgtctccaggggaaagagccaccctctcctgcagggccagtcagagtgttagcagcgacttagcctggtaccagcagaaacgtggccaggctcccaggctcctcatctatggtgcatccaccagggccactggtatcccagccaggttcagtggcagtgggtctgggacagagttcactctcaccatcagcagcctgcagtctgaagattttgcagtttattggtgtcagcagtataatgactggcctctcactttcggcggagggaccaaggtggagatcaac

SEQ ID NO: 3 - protein, 120 aa, Homo sapiens
  MISC_FEATURE: variable heavy chain - 122G3
QVQLVQSGAEVKKPGASVKVSCKASGYTFTNYAIHWVRQAPGQRLEWMGWINAGKGNTKYSQKFQGRVTITRDTSADTTYMELSSLRSEDTAVYFCAQAGLSSGLYRDYWGQGTPVTVSS

SEQ ID NO: 4 - protein, 107 aa, Homo sapiens
  MISC_FEATURE: variable light (kappa) chain - AB 122G3
EIVMTQSPATLSVSPGERATLSCRASQSVSSDLAWYQQKRGQAPRLLIYGASTRATGIPARFSGSGSGTEFTLTISSLQSEDFAVYWCQQYNDWPLTFGGGTKVEIN

SEQ ID NO: 5 - protein, 5 aa, Homo sapiens
  MISC_FEATURE: variable heavy chain - CDR1 - AB 122G3
NYAIH

SEQ ID NO: 6 - protein, 17 aa, Homo sapiens
  MISC_FEATURE: variable heavy chain - CDR2 - AB 122G3
WINAGKGNTKYSQKFQG

SEQ ID NO: 7 - protein, 11 aa, Homo sapiens
  MISC_FEATURE: variable heavy chain - CDR3 - AB 122G3
AGLSSGLYRDY

SEQ ID NO: 8 - protein, 11 aa, Homo sapiens
  MISC_FEATURE: variable light (kappa) chain - CDR1 - AB 122G3
RASQSVSSDLA

SEQ ID NO: 9 - protein, 7 aa, Homo sapiens
  MISC_FEATURE: variable light (kappa) chain - CDR2 - AB 122G3
GASTRAT

SEQ ID NO: 10 - protein, 7 aa, Homo sapiens
  MISC_FEATURE: variable light (kappa) chain - CDR3 - AB 122G3
QQYNDWP

SEQ ID NO: 11 - DNA, 360 bp, Homo sapiens
  misc_feature: variable heavy chain - 32H2
caggtgcagctggtggagtctgggggaggcgtggtccagcctgggaggtccctgagactctcctgtgcagcctctggattcaccttcagtagtaatgctatgcactgggtccgccaggctccaggcaaggggctggagtgggtggcagttatatcacatgatggaagcaataaatactatgcagactccgtgaagggccgattcaccatctccagagacaattccaagaacacgctgtatctgcaaatgaacagcctgagagctgaggacacggctgtgtattactgtgcgagagatggttcggggagttattttaatcttgactactggggccagggaaccctggtcaccgtctcctca

SEQ ID NO: 12 - DNA, 330 bp, Homo sapiens
  misc_feature: variable light (lambda) chain - AB 32H2
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctagtcttcggaactgggaccaaggtcaccgtccta

SEQ ID NO: 13 - protein, 120 aa, Homo sapiens
  MISC_FEATURE: variable heavy chain - 32H2
QVQLVESGGGVVQPGRSLRLSCAASGFTFSSNAMHWVRQAPGKGLEWVAVISHDGSNKYYADSVKGRFTISRDNSKNTLYLQMNSLRAEDTAVYYCARDGSGSYFNLDYWGQGTLVTVSS

SEQ ID NO: 14 - protein, 115 aa, Homo sapiens
  MISC_FEATURE: variable light (lambda) chain - AB 32H2
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLVFGTGTKVTVLSNAMH

SEQ ID NO: 15 - protein, 5 aa, Homo sapiens
  MISC_FEATURE: variable heavy chain - CDR1 - AB 32H2
SNAMH

SEQ ID NO: 16 - protein, 17 aa, Homo sapiens
  MISC_FEATURE: variable heavy chain - CDR2 - AB 32H2
VISHDGSNKYYADSVKG

SEQ ID NO: 17 - protein, 11 aa, Homo sapiens
  MISC_FEATURE: variable heavy chain - CDR3 - AB 32H2
DGSGSYFNLDY

SEQ ID NO: 18 - protein, 14 aa, Homo sapiens
  MISC_FEATURE: variable light (lambda) chain - CDR1 - AB 32H2
TGTSSDVGGYNYVS

SEQ ID NO: 19 - protein, 7 aa, Homo sapiens
  MISC_FEATURE: variable light (lambda) chain - CDR2 - AB 32H2
EVSNRPS

SEQ ID NO: 20 - protein, 10 aa, Homo sapiens
  MISC_FEATURE: variable light (lambda) chain - CDR3 - AB 32H2
SSYTSSSTLV

SEQ ID NO: 21 - DNA, 357 bp, Homo sapiens
  misc_feature: variable heavy chain - 34G9
gaggtgcaactggaggagtccgggggagacttagttcagcctggggggtccctgagactctcctgtgcagcctctggattcaccttcagtcactactggatgcactgggtccgccaagctccagggaaggggctggtttgggtctcacgtattaatagtgatggaagtagcacaagctacgcggactccgtgaagggccgattcaccatctccagagacaacgccaagaacacgctgtatctgcaaatgaacagtctgagagccgaggacacggctgtgtattactgtgcaaggataggtagcgatggctacaattatgacttctggggccagggaaccctggtcaccgtctcctca

SEQ ID NO: 22 - DNA, 339 bp, Homo sapiens
  misc_feature: variable light (kappa) chain - AB 34G9
gacatcgtgatgacccagtctccagactccctggctgtgtctctgggcgagagggccaccatcaactgcaagtccagccagagtgttttatacagctccaacaataagaactacttaacttggtaccagcagaaaccaggacagcctcctaagctgctcatttactgggcatctacccgggaatccggggtccctgaccgattcagtggcagcgggtctgggacagatttcactctcaccatcagcagcctgcaggctgaggatgtggcagtttattactgtcagcaatattatagtactcccgtggcgttcggccaagggaccaaggtggaaatcaaa

SEQ ID NO: 23 - protein, 119 aa, Homo sapiens
  MISC_FEATURE: variable heavy chain - 34G9
EVQLEESGGDLVQPGGSLRLSCAASGFTFSHYWMHWVRQAPGKGLVWVSRINSDGSSTSYADSVKGRFTISRDNAKNTLYLQMNSLRAEDTAVYYCARIGSDGYNYDFWGQGTLVTVSS

SEQ ID NO: 24 - protein, 113 aa, Homo sapiens
  MISC_FEATURE: variable light (kappa) chain - AB 34G9
DIVMTQSPDSLAVSLGERATINCKSSQSVLYSSNNKNYLTWYQQKPGQPPKLLIYWASTRESGVPDRFSGSGSGTDFTLTISSLQAEDVAVYYCQQYYSTPVAFGQGTKVEIK

SEQ ID NO: 25 - protein, 5 aa, Homo sapiens
  MISC_FEATURE: variable heavy chain - CDR1 - AB 34G9
HYWMH

SEQ ID NO: 26 - protein, 17 aa, Homo sapiens
  MISC_FEATURE: variable heavy chain - CDR2 - AB 34G9
RINSDGSSTSYADSVKG

SEQ ID NO: 27 - protein, 10 aa, Homo sapiens
  MISC_FEATURE: variable heavy chain - CDR3 - AB 34G9
IGSDGYNYDF

SEQ ID NO: 28 - protein, 17 aa, Homo sapiens
  MISC_FEATURE: variable light (kappa) chain - CDR1 - AB 34G9
KSSQSVLYSSNNKNYLT

SEQ ID NO: 29 - protein, 7 aa, Homo sapiens
  MISC_FEATURE: variable light (kappa) chain - CDR2 - AB 34G9
WASTRES

SEQ ID NO: 30 - protein, 9 aa, Homo sapiens
  MISC_FEATURE: variable light (kappa) chain - CDR3 - AB 34G9
QQYYSTPVA

SEQ ID NO: 31 - DNA, 357 bp, Homo sapiens
  misc_feature: variable heavy chain - AB 102G10
gaggtgcagctggtggagtctgggggaggcctggtcaagccgggggggtccctgagactctcctgtgcagcctctggattcaccttcagtgggaatagcatgaactgggtccgccaggctccagggaaggggctggagtgggtctcatccattagtagtagtagtggttacatatactacgcagactcagtgaagggccgattcaccgtctccagagacaacgccaagaactcactatatctgcaaatgaacagcctgagagccgaggacacggctgtgtattactgtgcgacctggccggtgactacggcgagggtcggctactggggccagggaaccctggtcaccgtctcctca

SEQ ID NO: 32 - DNA, 330 bp, Homo sapiens
  misc_feature: variable light (lambda) chain - AB 102G10
cagtctgtgctgactcagccaccctcagcgtctgggacccccgggcagagggtcaccatctcttgttctggaagcagctccaacatcggaagtaattatgtatactggcaccagcagctcccaggaacggcccccaaactcctcatctataggcatgatcagcggccctcaggggtccctgaccgattctctggctccaagtctggcacctcagcctccctggccatcagtgggctccggtccgaggatgaggctgattattactgtgcagcatgggatgacagcctgagtgcttgggtgttcggcggagggaccaagctgaccgtccta

SEQ ID NO: 33 - protein, 119 aa, Homo sapiens
  MISC_FEATURE: variable heavy chain - AB 102G10
EVQLVESGGGLVKPGGSLRLSCAASGFTFSGNSMNWVRQAPGKGLEWVSSISSSSGYIYYADSVKGRFTVSRDNAKNSLYLQMNSLRAEDTAVYYCATWPVTTARVGYWGQGTLVTVSS

SEQ ID NO: 34 - protein, 110 aa, Homo sapiens
  MISC_FEATURE: variable light (lambda) chain - AB 102G10
QSVLTQPPSASGTPGQRVTISCSGSSSNIGSNYVYWHQQLPGTAPKLLIYRHDQRPSGVPDRFSGSKSGTSASLAISGLRSEDEADYYCAAWDDSLSAWVFGGGTKLTVL

SEQ ID NO: 35 - protein, 5 aa, Homo sapiens
  MISC_FEATURE: variable heavy chain - CDR1 - AB 102G10
GNSMN

SEQ ID NO: 36 - protein, 17 aa, Homo sapiens
  MISC_FEATURE: variable heavy chain - CDR2 - AB 102G10
SISSSSGYIYYADSVKG

SEQ ID NO: 37 - protein, 10 aa, Homo sapiens
  MISC_FEATURE: variable heavy chain - CDR3 - AB 102G10
WPVTTARVGY

SEQ ID NO: 38 - protein, 13 aa, Homo sapiens
  MISC_FEATURE: variable light (lambda) chain - CDR1 - AB 102G10
SGSSSNIGSNYVY

SEQ ID NO: 39 - protein, 7 aa, Homo sapiens
  MISC_FEATURE: variable light (lambda) chain - CDR2 - AB 102G10
RHDQRPS

SEQ ID NO: 40 - protein, 11 aa, Homo sapiens
  MISC_FEATURE: variable light (lambda) chain - CDR3 - AB 102G10
AAWDDSLSAWV

SEQ ID NO: 41 - protein, 314 aa, Homo sapiens
  MISC_FEATURE: Human MAGE-A3
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 42 - protein, 60 aa, Homo sapiens
  MISC_FEATURE: aa 31 to 90 of MAGE-A3
PATEEQEAASSSSTLVEVTLGEVPAAESPDPPQSPQGASSLPTTMNYPLWSQSYEDSSNQ

SEQ ID NO: 43 - protein, 20 aa, Homo sapiens
  MISC_FEATURE: aa 151 to 170 of MAGE-A3
FSKASSSLQLVFGIELMEVD

SEQ ID NO: 44 - protein, 20 aa, Homo sapiens
  MISC_FEATURE: aa 231 to 250 of MAGE-A3
FEGREDSILGDPKKLLTQHF

SEQ ID NO: 45 - protein, 30 aa, Homo sapiens
  MISC_FEATURE: aa 61 to 90 of MAGE-A3
PPQSPQGASSLPTTMNYPLWSQSYEDSSNQ

SEQ ID NO: 46 - protein, 30 aa, Homo sapiens
  MISC_FEATURE: aa 31 to 60 of MAGE-A3
PATEEQEAASSSSTLVEVTLGEVPAAESPD

SEQ ID NO: 47 - protein, 20 aa, Homo sapiens
  MISC_FEATURE: aa 51 to 70 of MAGE-A3
GEVPAAESPDPPQSPQGASS

SEQ ID NO: 48 - protein, 20 aa, Homo sapiens
  MISC_FEATURE: aa 41 to 60 of MAGE-A3
SSSTLVEVTLGEVPAAESPD

SEQ ID NO: 49 - protein, 112 aa, Homo sapiens
  MISC_FEATURE: human variable light (kappa) chain - 12D7 (NY-ESO-1)
DIVMTQTPLSLPVTLGQPASLSCRSSQSLVFTDGNTYLNWFQQRPGQSPRRLIYKVSSRDPGVPDRFSGTGSGTDFTLEISRVEAEDIGVYYCMQGTHWPPIFGQGTKVEIK

SEQ ID NO: 50 - protein, 118 aa, Homo sapiens
  MISC_FEATURE: human variable heavy chain - 12D7 (NY-ESO-1)
QVQLVQSGGGVVRPGGSLRLSCAASGFSFIDYGMSWVRQVPGKGLEWVAGMNWSGDKKGHAESVKGRFIISRDNAKNTLYLEMSSLRVEDTALYFCARGEYSNRFDPRGRGTLVTVSS